(19) United States
(12) Patent Application Publication
(10) Pub. No.: US 2023/0414945 A1
(43) Pub. Date: Dec. 28, 2023

(54) SLEEP APNEA TREATMENT SYSTEM AND METHOD

(71) Applicant: Capri Medical Limited, Dublin (IE)

(72) Inventors: Fergal Ward, Dublin 4 (IE); Ross Lewis, Dublin 4 (IE); Ricardo Vallejo, Hallandale Beach, FL (US); Jorge Armando Cuartas, Envigado (CO); Michael Herzog, Cottbus (DE); David Leonardo Cedeno, Parkland, FL (US); Willard S. Noyes, Bloomington, IL (US)

(73) Assignee: Capri Medical Limited, Dublin (IE)

(21) Appl. No.: 18/198,750

(22) Filed: May 17, 2023

Related U.S. Application Data
(60) Provisional application No. 63/354,463, filed on Jun. 22, 2022.

(51) Int. Cl. A61N 1/36; A61B 5/00; A61B 5/0205; A61M 16/06

(52) U.S. Cl. CPC A61N 1/3611; A61N 1/36139; A61B 5/4818; A61B 5/02055; A61M 16/06

(57) ABSTRACT

Disclosed is a method of treating sleep apnea in a patient by operating an implanted neurostimulator implant to stimulate the genioglossus nerve branch and/or the genioglossus muscle. Also disclosed is a method of implanting a neurostimulator implant in the genioglossus muscle proximal to the genioglossus nerve branch. Further disclosed is a sleep apnea treatment system and a guide for the sleep apnea treatment system.

### CROSS REFERENCE TO RELATED APPLICATIONS

This application claims the benefit of U.S. Provisional Application Ser. No. 63/354,463, filed Jun. 22, 2022, and entitled “METHOD AND APPARATUS FOR TREATING PHYSIOLOGICAL CONDITIONS USING INJECTABLE ELECTRODES.”

This application also claims the benefit of the following U.S. Provisional Applications filed by the same applicant on an even date herewith, the entire contents of which are incorporated herein by this reference for all purposes:
U.S. Provisional Application No. 63/467,117, filed May 17, 2023, entitled “WIRELESS POWER TRANSFER IN MEDICAL IMPLANTS,” Attorney Docket No. 58771-00030;
U.S. Provisional Application No. 63/467,044, filed May 17, 2023, entitled “WIRELESS POWER TRANSFER IN MEDICAL IMPLANTS,” Attorney Docket No. 58771-00035;
U.S. Provisional Application No. 63/467,113, filed May 17, 2023, entitled “SYSTEMS FOR MONITORING, DIAGNOSING AND/OR DELIVERING THERAPEUTIC TREATMENT,” Attorney Docket No. 58771-00040;
U.S. Provisional Application No. 63/467,081, filed May 17, 2023, entitled “TELEMETRY SYSTEM,” Attorney Docket No. 58771-00045;
U.S. Provisional Application No. 63/467,089, filed May 17, 2023, entitled “RF TRANSCEIVER” Attorney Docket No. 58771-00050; and
U.S. Provisional Application No. 63/467,069, filed May 17, 2023, entitled “MEDICAL IMPLANT,” Attorney Docket No. 58771-00055.
Further, the entire contents of the following U.S. non-provisional applications, filed by the same applicant on an even date herewith, are incorporated herein by this reference for all purposes:
U.S. patent application Ser. No. ______, filed May 17, 2023, entitled “METHOD OF PERCUTANEOUSLY IMPLANTING A MEDICAL IMPLANT AND A DELIVERY DEVICE FOR PERCUTANEOUS DELIVERY OF A MEDICAL IMPLANT,” Attorney Docket No. 58771-00006; and
U.S. patent application Ser. No. ______, filed May 17, 2023, entitled “DELIVERY DEVICE AND METHOD FOR PERCUTANEOUSLY IMPLANTING A MEDICAL IMPLANT,” Attorney Docket No. 58771-0011.

### BACKGROUND

There exist a number of treatments for sleep apnea, such as continuous positive airway pressure devices (CPAP), surgically implanted neurostimulators, oral mouthpieces and medicines. Generally, known devices have poor efficacy with respect to reducing apnea-hypopnea index (AHI) and/or they are highly invasive devices procedures that can only be performed by surgical physicians. Additionally, neurostimulators and positive airway pressure (PAP) devices on the market act primarily as standalone devices, capable of partially treating only OSA or CSA.

US2022370797A1 discloses techniques for implanting a signal delivery device to treat sleep apnea. Specifically, US2022370797A1 teaches a method of percutaneous implantation of a signal delivery device to target the medial branches of the Hypoglossal nerve. As described in US2022370797A1, stimulation of the medial branches of the Hypoglossal nerve can innervate oral cavity muscles such as the genioglossus and geniohyoid muscles, which tend to pull the tongue forward (anteriorly), thus reducing the tendency for the soft tissue of the palate to prolapse into the patient's airway. However, the medial branch also includes retrusers which innervate muscles such as the styloglossus and the hyoglossus muscles, which tend to pull the soft tissue backward (posteriorly), and/or can cause the tongue to curl left or right within the mouth—both are motor responses that can obstruct the patient's airway. Accordingly, it is important to stimulate the medial branch in a manner that results in a net positive protrusive effect or a net protrusive motor response. This can include, for example, stimulating the medial branch so as to avoid activating the retrusers entirely.

A problem with the methods taught in US2022370797A1 is that the implant position relative to the medial branch of the Hypoglossal nerve, and the electrical stimulating signal, will determine the balance of protrusive-retrusive stimulation. Accordingly, the implantation procedure requires great accuracy and calibration, resulting in a relatively complex and intrusive procedure, typically requiring surgical interventions such as use of an endoscope during calibration.

US20130218230A1 discloses a method of stimulating a Hypoglossal nerve for controlling the position of a patient's tongue, for example to prevent obstructive sleep apnea. US20130218230A1 suggests that stimulating the distal branches of the Hypoglossal nerve may generate only the desired tongue movements by targeting only some of the tongue muscles, particularly those that cause tongue protraction.

As explained in US20130218230A1, one problem with targeting the distal branches of the Hypoglossal nerve is that the surgical approach to these distal branches is more difficult and the branches are progressively smaller the more distal the placement of the electrode, making the design of an appropriate electrode for such small branches more difficult and the systems used to stimulate them less robust and the opportunity for damage for these more delicate structures more likely. It also makes the implantation procedure difficult.

Accordingly, it would be advantageous to provide an implantable neurostimulator system that targets the distal branches of the Hypoglossal nerve, and an implantation procedure that addresses the difficulties described above.

### BRIEF SUMMARY

According to the present disclosure there is provided a method of treating sleep apnea in a patient, the method comprising operating an implanted neurostimulator implant to stimulate a branch of the hypoglossal nerve. In examples, the implanted neurostimulator implant is operable to stimulate a distal branch of the hypoglossal nerve, in particular the genioglossus nerve branch. In other examples, the implanted neurostimulator implant is operable to stimulate a medial branch of the hypoglossal nerve.

In examples, the method comprises operating the implanted neurostimulator implant to stimulate the genioglossus nerve branch and/or the genioglossus muscle. Stimulating the genioglossus nerve branch and/or the genioglossus muscle may cause protraction of the tongue, alleviating sleep apnea.

In examples, the implanted neurostimulator implant is operable to directly stimulate the genioglossus nerve branch and/or the genioglossus muscle. Such direct stimulation may be provided by implanting an electrode of the neurostimulator implant in the genioglossus muscle, in close proximity with the genioglossus nerve branch. By implanting the electrode in close proximity with the genioglossus nerve branch, for example within 2 cm, or within 1 cm of the genioglossus nerve branch, the amplitude of the stimulation signal can be limited such that only the genioglossus nerve branch is stimulated. This can avoid stimulation of other parts of the hypoglossal nerve and other muscles and thereby avoid retrusion of the tongue. In examples, the implanted neurostimulator implant is operable to only stimulate the genioglossus nerve branch and/or the genioglossus muscle. In examples, the implanted neurostimulator implant is operable to not stimulate the medial branches of the hypoglossal nerve.

In examples, the implanted neurostimulator implant is operated to stimulate the genioglossus nerve branch with a first stimulation signal and to stimulate the genioglossus muscle with a second stimulation signal. The first and second stimulation signals may be generated simultaneously or sequentially. The first and second stimulation signals may be generated in response to a detected physiological parameter or movement of the patient, as described further below.

In examples, the implanted neurostimulator implant is operated to stimulate the genioglossus muscle with a stimulation signal having a frequency of between about 2 Hz and about 150 Hz, for example between about 2 Hz and about 100 Hz, for example between about 2 Hz and about 50 Hz, for example between about 2 Hz and about 40 Hz, for example between about 2 Hz and about 30 Hz, for example between about 2 Hz and about 20 Hz, for example between about 2 Hz and about 15 Hz, for example between about 2 Hz and about 10 Hz.

In examples, the stimulation signal stimulating the genioglossus muscle has a peak amplitude of between 0.1 mA and 10 mA, for example between 0.1 mA and 5 mA. Such low frequency, low amplitude muscle stimulation may avoid discomfort of the patient.

In examples, the implanted neurostimulator implant is operated to stimulate the genioglossus nerve branch with a stimulation signal having a frequency of between about 20 Hz and about 1500 Hz, for example between about 20 Hz and about 1000 Hz, for example between about 20 Hz and about 500 Hz, for example between about 20 Hz and about 400 Hz, for example between about 20 Hz and about 300 Hz, for example between about 20 Hz and about 200 Hz, for example between about 20 Hz and about 150 Hz, for example between about 20 Hz and about 100 Hz.

In examples, the stimulation of the genioglossus nerve branch and/or the genioglossus muscle comprises a stimulation signal having a plurality of pulses, each pulse having a pulse width of between 30 microseconds and 2,000 microseconds. In examples, the pulse interval may be between 30 microseconds and 2,000 microseconds.

In examples, an electrode providing the stimulation signal to a nerve or muscle may be operated as an anode or as a cathode. The neurostimulator implant may be controllable to change between anodic stimulation and cathodic stimulation.

In examples, the neurostimulator implant may comprise an electrode lead having an electrode. The method may comprise implanting the electrode lead in the genioglossus muscle proximal to the genioglossus nerve branch of the patient. Accordingly, the neurostimulator implant can directly stimulate the genioglossus nerve branch within the genioglossus muscle. In examples, the electrode may be implanted to within about 2 cm of the genioglossus nerve branch, preferably within about 1 cm of the genioglossus nerve branch.

In examples, the neurostimulator implant may further comprise a housing portion. The electrode lead may extend from the housing portion. The method may comprise implanting the housing portion in a subcutaneous tissue of the patient.

In examples, the method may further comprise transferring wireless power from an external device to the implanted neurostimulator implant. In examples, the neurostimulator implant comprises a wireless power receiver (e.g., antenna). The wireless power receiver may be provided in the housing portion and/or in the electrode lead. In examples, the wireless power transferred to the implanted neurostimulator implant comprises a frequency of between 300 MHz and 3 GHz, for example between 400 MHz and 2.5 GHz, for example between 433 MHz and 2.4 GHz.

In examples, the method may further comprise detecting, by a sensor, at least one physiological parameter. In examples, the at least one physiological parameter may comprise at least one of:
a respiration rate of the patient,
an electromyograph, EMG, of the patient,
an electrocardiogram, ECG, of the patient,
an oxygen saturation of the patient,
a body temperature of the patient,
a heart rate of the patient,
a blood pressure of the of the patient.

In examples, the method may comprise generating a stimulation signal in response to the detected at least one physiological parameter. The method may comprise adapting the stimulation signal based on the detected at least one physiological parameter.

In examples, the method may further comprise detecting, by a sensor, at least one movement of the patient, for example at least one of:
a respiratory movement of the patient,
a mandibular movement of the patient,
a thoracic movement of the patient,
a diaphragm movement of the patient.

In examples, the method may comprise generating a stimulation signal in response to the detected at least one movement. The method may comprise adapting the stimulation signal based on the detected at least one movement.

In examples, the method may further comprise detecting, by a sensor, a physiological parameter or movement of the patient indicative of sleep apnea, for example at least one of:
a lapse is respiratory rate,
an electromyograph, EMG, profile indicative of sleep apnea,
an electrocardiogram, ECG, profile indicative of sleep apnea,
a decrease in oxygen saturation,
an increase in body temperature,
an increase in heart rate,
an increase in blood pressure, or
a respiratory, mandibular, thoracic or diaphragm movement.

In examples, the method may comprise generating a stimulation signal in response to the detected at least one physiological parameter or movement of the patient indicative of sleep apnea. The method may comprise adapting the stimulation signal based on the detected at least one physiological parameter or movement of the patient indicative of sleep apnea.

In examples, the method may further comprise controlling the neurostimulator implant to stimulate the genioglossus nerve branch and/or the genioglossus muscle in response to the detected at least one physiological parameter of the patient and/or the detected at least one movement of the patient and/or the at least one physiological parameter or movement of the patient indicative of sleep apnea. Accordingly, the neurostimulator implant can be operated to provide stimulation at a time determined based on the parameter detected by the sensor, and/or with a stimulation signal configured based on the parameter detected by the sensor. The stimulation signal may be configured with specific period and/or frequency and/or amplitude and/or duration and/or pulse width and/or pulse interval in response to the parameter detected by the sensor. The stimulation signal may be generated for stimulating the genioglossus nerve branch and/or the genioglossus muscle.

In one example, the method comprises detecting, by a sensor, a respiratory rate of the patient, and stimulating the genioglossus nerve branch and/or the genioglossus muscle synchronously with the detected respiratory rate. In another example, the method comprises stimulating the genioglossus nerve branch and/or the genioglossus muscle synchronously with a predetermined target respiratory rate. In examples, the method may comprise stimulating the genioglossus nerve branch and/or the genioglossus muscle at the start of an inhalation cycle of the patient.

In examples, the method may further comprise determining, from the detected at least one physiological parameter and/or at least one movement of the patient, a classification of sleep apnea, in particular whether the detected sleep apnea comprises obstructive sleep apnea, OSA, central sleep apnea, CSA, or a combination of OSA and CSA. The method may include adapting the stimulation signal based on the determined classification of sleep apnea.

In examples, the method may further comprise operating a second implanted neurostimulator implant to stimulate a phrenic nerve and/or an ansa cervicalis nerve of the patient. Stimulation of the phrenic nerve and/or an ansa cervicalis may be in response to a detected physiological parameter, patient movement, or classification of sleep apnea as described above. The stimulation of the phrenic nerve and/or an ansa cervicalis may be in response to a detected apneic event. The stimulation of the phrenic nerve and/or an ansa cervicalis may be synchronized with the stimulation of the genioglossus nerve branch and/or the genioglossus muscle.

In examples, the method may further comprise operating a continuous positive air pressure, CPAP, device to provide positive air pressure to the patient's airways. Operation of the CPAP device may be in response to a detected physiological parameter, patient movement, or classification of sleep apnea as described above. The operation of the CPAP device may be in response to a detected apneic event. The operation of the CPAP device may be synchronized with the stimulation of the genioglossus nerve branch and/or the genioglossus muscle.

In examples, the method may further comprise controlling the or each neurostimulator implant, and optionally the CPAP device, based on the detected at least one physiological parameter of the patient and/or the detected at least one movement of the patient. In examples, the method may comprise operating the neurostimulator implant and/or the second neurostimulator implant and/or the CPAP device based on the determined classification of sleep apnea. In examples, the method may comprise, if the classification of sleep apnea is determined to be obstructive sleep apnea, OSA, operating the neurostimulator device to stimulate the genioglossus nerve branch and/or the genioglossus muscle. In examples, if the classification of sleep apnea is determined to be central sleep apnea, CSA, the method may comprise operating the second neurostimulator implant to stimulate the phrenic nerve and/or ansa cervicalis nerve. In examples, if the classification of sleep apnea is determined to be obstructive sleep apnea, OSA, the method may comprise operating the CPAP device to provide positive air pressure to the patient. In examples, if the classification of sleep apnea is determined to be a combination of obstructive sleep apnea and central sleep apnea, the method may comprise operating:
the neurostimulator device to stimulate the genioglossus nerve branch and genioglossus muscle, and
at least one of:
the second neurostimulator implant to stimulate the phrenic nerve and/or ansa cervicalis nerve,
and the CPAP device to provide positive air pressure to the patient.

In examples, the sensor may be integrated with the neurostimulator implant. In other examples, the sensor may be an external sensor. In examples, there may be more than one sensor arranged to detect more than one physiological parameter or movement of the patient, and each sensor may be integrated with the neurostimulator implant or an external device. In one example, the neurostimulator implant comprises an oxygen saturation sensor and/or an ECG sensor, and an external accelerometer is provided to detect a movement of the patient, such as mandibular or head movement.

According to a further aspect of the present disclosure there is also provided a system for treating sleep apnea of a patient, the system comprising a neurostimulator implant implantable proximal to a branch of the hypoglossal nerve of the patient and operable to stimulate the branch of the hypoglossal nerve.

In examples, the implanted neurostimulator implant is operable to stimulate the genioglossus nerve branch and/or the genioglossus muscle of the patient. In other examples, the implanted neurostimulator implant is operable to stimulate the medial branches of the hypoglossal nerve.

In examples, the implanted neurostimulator implant is operable to stimulate the genioglossus muscle with a stimulation signal having a frequency of between about 2 Hz and about 150 Hz, for example between about 2 Hz and about 100 Hz, for example between about 2 Hz and about 50 Hz, for example between about 2 Hz and about 40 Hz, for example between about 2 Hz and about 30 Hz, for example between about 2 Hz and about 20 Hz, for example between about 2 Hz and about 15 Hz, for example between about 2 Hz and about 10 Hz.

In examples, the stimulation signal stimulating the genioglossus muscle has a peak amplitude of between 0.1 mA and 10 mA, for example between 0.1 mA and 5 mA. Such low frequency, low amplitude muscle stimulation may avoid discomfort of the patient.

In examples, the implanted neurostimulator implant is operable to stimulate the genioglossus nerve branch with a stimulation signal having a frequency of between about 20 Hz and about 1500 Hz, for example between about 20 Hz and about 1000 Hz, for example between about 20 Hz and about 500 Hz, for example between about 20 Hz and about 400 Hz, for example between about 20 Hz and about 300 Hz, for example between about 20 Hz and about 200 Hz, for example between about 20 Hz and about 150 Hz, for example between about 20 Hz and about 100 Hz.

In examples, the neurostimulator implant is operable to stimulate the genioglossus nerve branch and/or the genioglossus muscle with a stimulation signal having a plurality of pulses, each pulse having a pulse width of between 30 microseconds and 2,000 microseconds. In examples, the pulse interval may be between 30 microseconds and 2,000 microseconds.

In examples, the neurostimulator implant comprises an electrode that is implantable within the genioglossus muscle proximal to a genioglossus nerve branch of the patient, and a signal generator for generating a stimulation signal to stimulate the genioglossus nerve branch and/or the genioglossus muscle of the patient. In examples, the neurostimulator implant is implantable within the genioglossus muscle in close proximity with the genioglossus nerve branch for direct stimulation of the genioglossus muscle and/or the genioglossus nerve branch.

In examples, the neurostimulator implant is a battery-less implant and comprises a wireless power receiver. In examples, the system further comprises an external device. The external device may comprise a wireless power transmitter. The wireless power transmitter is for transmitting wireless power to the neurostimulator implant when the neurostimulator implant is implanted in the genioglossus muscle. The neurostimulator implant may comprise a wireless power antenna for receiving wireless power. In some examples, the external device comprises a signal generator that generates a stimulation signal. In other examples, a signal generator is provided in the neurostimulator implant.

In examples, the system may further comprise a sensor arranged to detect at least one physiological parameter of the patient and/or at least one movement of the patient.

In examples, the at least one physiological parameter comprises one or more of:
a respiration rate of the patient,
an electromyograph, EMG, of the patient,
an electrocardiogram, ECG, of the patient,
an oxygen saturation of the patient,
a body temperature of the patient,
a heart rate of the patient,
a blood pressure of the of the patient.

In examples, the stimulation signal generated by the signal generator may be generated in response to the detected at least one physiological parameter. The stimulation signal may be adapted based on the detected at least one physiological parameter.

In examples, the at least one movement of the patient comprises at least one of:
a respiratory movement of the patient,
a mandibular movement of the patient,
a thoracic movement of the patient,
a diaphragm movement of the patient.

In examples, the stimulation signal generated by the signal generator may be generated in response to the detected at least one movement of the patient. The stimulation signal may be adapted based on the detected at least one movement of the patient.

In examples, the system may further comprise a controller configured to determine the onset or occurrence of sleep apnea based on the detected physiological parameter and/or the detected movement of the patient, for example at least one of:
a lapse is respiratory rate,
an electromyograph, EMG, profile indicative of sleep apnea,
an electrocardiogram, ECG, profile indicative of sleep apnea,
a decrease in oxygen saturation,
an increase in body temperature,
an increase in heart rate,
an increase in blood pressure, or
a respiratory, mandibular, thoracic or diaphragm movement.

In examples, the stimulation signal generated by the signal generator may be generated in response to the detected onset or occurrence of sleep apnea. The stimulation signal may be adapted based on the detected onset or occurrence of sleep apnea.

In examples, the signal generator may be configured to generate the stimulation signal in response to the detected at least one physiological parameter of the patient and/or the detected at least one movement of the patient and/or the at least one physiological parameter or movement of the patient indicative of sleep apnea. Accordingly, the neurostimulator implant can be operated to provide stimulation at a time determined based on the parameter detected by the sensor, and/or with a stimulation signal configured based on the parameter detected by the sensor. The stimulation signal may be adapted or configured with specific period and/or frequency and/or amplitude and/or duration and/or pulse width and/or pulse interval in response to the parameter detected by the sensor.

In examples, the system may further comprise a second neurostimulator implant comprising:
a neurostimulator implant comprising an electrode,
a signal generator for generating a stimulation signal, and
a wireless power system for providing power to the neurostimulator implant,
wherein the electrode is implantable to stimulate a phrenic nerve and/or an ansa cervicalis nerve of the patient.

In examples, the system may further comprise a continuous positive air pressure, CPAP, device operable to provide positive air pressure to the patient.

In examples, the system may further comprise a controller configured to control the or each neurostimulator implant, and optionally the CPAP device, based on the detected at least one physiological parameter of the patient and/or the detected at least one movement of the patient. The controller may be provided in one of the neurostimulator implant, the CPAP device, or an external device.

In examples, the controller may be configured to determine, based on the detected at least one physiological parameter and/or the at least one movement of the patient, a classification of sleep apnea, in particular whether the detected sleep apnea comprises obstructive sleep apnea, OSA, central sleep apnea, CSA, or a combination of OSA and CSA.

In examples, the controller may be configured to operate the neurostimulator implant and/or the second neurostimulator implant and/or the CPAP device based on the determined classification of sleep apnea.

According to a further aspect of the present disclosure there is also provided a method of implanting a neurostimulator implant in a patient for treating sleep apnea, wherein the neurostimulator implant comprises a housing portion and a flexible elongate electrode lead extending from the housing portion and having an electrode, the method comprising:
providing a delivery device having a first needle holding the housing portion of the neurostimulator implant and a second needle holding the electrode lead, wherein the second needle has a higher gauge than the first needle, wherein the second needle extends beyond the first needle, and wherein the second needle is retractable in a direction towards the first needle,
inserting the second needle into the patient at an insertion point under the patient's chin,
progressing the second needle through the patient's tissue along an insertion direction towards the genioglossus muscle of the patient,
when the second needle is within the genioglossus muscle of the patient, retracting the second needle to implant the electrode lead with the electrode proximal to a genioglossus nerve branch within the genioglossus muscle, and
ejecting the housing portion from the first needle to implant the housing portion underneath the skin.

In examples, the insertion point is proximal to the posterior edge of the patient's mandibular symphysis.

In examples, the insertion direction comprises a first angle, β, in a mandibular plane of the patient's chin, and a second angle, θ, in the sagittal plane of the patient. In examples, the first angle, β, is between about ±15 degrees and about ±30 degrees, for example between about ±18 degrees and about ±25 degrees, for example between about ±20 degrees and about ±23 degrees, for example about ±22 degrees, in particular about ±21.8 degrees. In examples, the second angle is between about 40 degrees and about 65 degrees, for example between about 45 degrees and about 55 degrees, for example between about 40 degrees and about 50 degrees, for example between about 47 degrees and about 49 degrees, in particular about 48.1 degrees.

In examples, the method comprises scanning the patient and determining the insertion direction based on the scan.

In examples, the method further comprises rotating the delivery device before ejecting the housing portion from the first needle such that the housing portion is implanted in a different orientation to the electrode lead. In examples, the delivery device is rotated towards a position in which the first needle is parallel with the skin surface and with a tip of the first needle positioned underneath the skin. In examples, rotating the delivery device causes the electrode lead to bend. In examples, the first needle comprises a slot, and wherein a part of the electrode lead passes through the slot as the delivery device is rotated.

In examples, the method further comprises using an ultrasound device to monitor the position of the second needle.

In examples, the method further comprises positioning a guide on the patient, the guide having a guide channel that defines the insertion direction for the second needle, and positioning the second needle in the guide channel. In examples, the method further comprises removing the guide while the second needle is percutaneously positioned.

According to a further aspect of the present disclosure there is also provided a sleep apnea treatment system comprising:
a neurostimulator implant having an electrode;
a delivery device for implanting the neurostimulator implant such that the electrode is implanted in the genioglossus muscle of the patient proximal to a genioglossus nerve branch; and
a guide positionable on the patient and comprising a guide channel configured to receive a part of the delivery device and defining an insertion direction for the delivery device to implant the neurostimulator implant in the genioglossus muscle in proximity with the genioglossus nerve branch.

In examples, the neurostimulator implant comprises a housing portion and a flexible elongate electrode lead extending from the housing portion. In examples, the delivery device comprises a first needle adapted to hold the housing portion of the neurostimulator implant and a second needle adapted to hold the electrode lead, the second needle having a higher gauge than the first needle and extends beyond the first needle.

In examples, the second needle is retractable in a direction towards the first needle for implanting the electrode lead.

In examples, the guide channel is moveable to align the guide channel with an insertion point on the patient. In examples, the guide channel is rotatable to alter the insertion direction. Accordingly, the guide can be adjusted to the specific anatomy of the patient.

In examples, the guide further comprises an attachment for an ultrasound scanner. The ultrasound scanner can be attached to the guide such that an ultrasound sensor window is directed towards the implantation site for monitoring the position of the neurostimulator implant during implantation.

In examples, the guide channel comprises a slot for removal of the guide while the delivery device is percutaneously implanted. Accordingly, the delivery device can be rotated during implantation, for example after implantation of the electrode.

In examples, the guide comprises a locating portion positionable against the patient. In examples, the locating portion is shaped to be positioned on a chin of the patient. In examples, the locating portion is shaped to be received in the patient's mouth. In examples, the locating portion is configured to engage the patient's mandible. In examples, the locating portion comprises a brace configured to engage the patient's mandible and the patient's neck and/or shoulder.

According to a further aspect of the present disclosure there is provided a guide for a delivery device for percutaneous implantation of a neurostimulator implant in the genioglossus muscle of a patient proximal to a genioglossus nerve branch, wherein the guide comprises:
a locating portion positionable against the patient; and
a guide channel configured to receive a part of the delivery device and defining an insertion direction for the delivery device to implant the neurostimulator implant in the genioglossus muscle in proximity with the genioglossus nerve branch.

In examples, the delivery device comprises a first needle adapted to hold the housing portion of the neurostimulator implant and a second needle adapted to hold the electrode lead, and wherein the guide channel is configured to receive the second needle.

In examples, the guide channel is moveable to align the guide channel with an insertion point on the patient. In examples, the guide channel is rotatable to alter the insertion direction.

In examples, the guide further comprises an attachment for an ultrasound scanner.

In examples, the guide channel comprises a slot for removal of the guide while the delivery device is percutaneously implanted.

In examples, the locating portion is shaped to be positioned on a chin of the patient. In examples, the locating portion is shaped to be received in the patient's mouth. In examples, the locating portion is configured to engage the patient's mandible. In examples, the locating portion comprises a neck brace configured to engage the patient's mandible and the patient's neck.

According to an aspect of the disclosure, there is provided a method of percutaneously implanting one or more neurostimulator implants such that an electrode of the neurostimulator implant is implanted proximal to a genioglossus nerve branch, in particular a genioglossus horizontal nerve branch. The percutaneous implantation avoids the submandibular gland and conducts and vascular structures. The neurostimulator implant can be implanted with combined anatomical landmarks and ultrasound guidance.

According to another aspect of the disclosure, integrated biosensors in the neurostimulator implant may detect signs of inspiration to trigger device activation and or general patient diagnostics. Such biosensors may include bioimpedance or biopotential based sensing devices whose respective signals are converted to ECG, EMG, EDA formats for interpreting heart rate, blood pressure and/or electromyographic detection of inspiratory muscle contraction. The neurostimulator implant may communicate with an external wearable device or an external power unit to combine data sets and/or trigger device activation in synchronisation with the patient's natural respiration rate.

According to an aspect of the disclosure, there is provided a method of treating a patient, comprising: A) detecting a physiological parameter of the patient, and B) activating a neural stimulation in synchronisation with the patient's respiration rate. In one example, the method includes detecting the patient's respiration rate. In one example, the method comprises stimulation of the hypoglossal nerve of the patient, in particular a genioglossus nerve branch, in synchronisation with the patient's respiration rate.

According to another aspect of the disclosure, integrated biosensors in a wearable device are used to detect signs of inspiration to trigger device activation and or general patient diagnostics. Such biosensors may include bioimpedance or biopotential based sensing devices whose respective signals are converted to ECG, EMG, EDA formats for interpreting heart rate, blood pressure and/or electromyographic detection of inspiratory muscle contraction but also oxygen, carbon dioxide, (to detect signs of hypoxia or anoxia) and the use of a microphone and/or an accelerometer to detect signs of airway obstruction.

### BRIEF DESCRIPTION OF THE DRAWINGS

To easily identify the discussion of any particular element or act, the most significant digit or digits in a reference number refer to the figure number in which that element is first introduced.

FIG. 1 illustrates the anatomical location of the hypoglossal nerve of a patient.

FIG. 2 illustrates an example neurostimulator implant for treating sleep apnea.

FIG. 3A illustrates a side view of a first example implant location for the neurostimulator implant of FIG. 2.

FIG. 3B illustrates a front view of the example implant location of FIG. 3A.

FIG. 4 illustrates a front view of a second example implant location with two neurostimulator implants.

FIG. 5A illustrates a front view of a third example implant location, with three neurostimulator implants.

FIG. 5B illustrates a side view of the third example implant location of FIG. 5A.

FIG. 6 illustrates a front view of a fourth example implant location with four neurostimulator implants.

FIG. 7 illustrates an example delivery device for implanting the neurostimulator implant of FIG. 2.

FIG. 8 illustrates a cross-sectional view of the delivery device of FIG. 7.

FIG. 9A illustrates the first and second needles of the delivery device of FIG. 7.

FIG. 9B illustrates a cross-sectional view of FIG. 9A.

FIG. 10A to FIG. 10L illustrate a method of implanting the neurostimulator implant of FIG. 2 using the delivery device of FIG. 7 to target the genioglossus nerve branch of the hypoglossal nerve of the patient.

FIG. 11 illustrates the electrode lead of the neurostimulator implant and the first and second needles of the delivery device during the implantation method.

FIG. 12A illustrates an example insertion point and insertion orientation for implanting the neurostimulator implant.

FIG. 12B illustrates a bottom view of the insertion point and insertion orientation shown in FIG. 12A.

FIG. 12C illustrates a side view of the insertion point and insertion orientation shown in FIG. 12A.

FIG. 12D illustrates the mandibular and muscular planes of a patient.

FIG. 12E illustrates measurement of the main distance of a patient using an image.

FIG. 13A illustrates an example guide for use in the implantation method of FIG. to FIG. 10 L.

FIG. 13B illustrates detail of the example guide of FIG. 13A.

FIG. 14A illustrates use of the example guide of FIG. 13A and FIG. 13B with the delivery device of FIG. 7.

FIG. 14B illustrates use of the example guide of FIG. 13A and FIG. 13B with the delivery device of FIG. 7.

FIG. 15A illustrates use of the example guide of FIG. 13A and FIG. 13B with the delivery device of FIG. 7.

FIG. 15B illustrates use of the example guide of FIG. 13A and FIG. 13B with the delivery device of FIG. 7.

FIG. 16 illustrates a guide having a brace.

FIG. 17 illustrates an example guide.

FIG. 18 illustrates a further example guide.

FIG. 19 illustrates a method of operating a sleep apnea treatment system.

### DETAILED DESCRIPTION

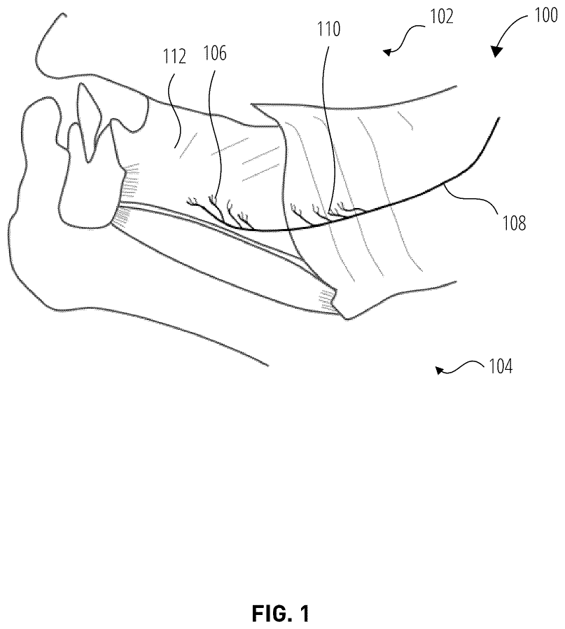

FIG. 1 illustrates parts of the nervous system in the area of the head 102 and neck 104 of a patient 100. As illustrated, the left hypoglossal nerve 108 is a motor nerve originating from the medulla obligate of the brain stem and extends towards the chin of the patient 100. The hypoglossal nerve 108 innervates all the extrinsic and intrinsic muscles of the tongue, except for the palatoglossus. The hypoglossal nerve 108 includes medial branches 110 and more distal branches. The medial branches of the hypoglossal nerve 108 innervate oral cavity muscles such as the genioglossus muscle 112, and also the geniohyoid, styloglossus and hyoglossus muscles. Some of these muscles cause protrusion of the tongue, and other cause retrusion of the tongue. The most distal branches of the hypoglossal nerve 108 form the genioglossus nerve branch 106, which innervates the genioglossus muscle 112. Contraction of the genioglossus muscle 112 causes tongue protrusion. It will be appreciated that a right hypoglossal nerve is present on the opposite side and has the same anatomy.

The disclosure, as set out below, provides a neurostimulator implant that is implantable to target the genioglossus nerve branch 106 within the genioglossus muscle 112. The difficulties of providing such an implant is that the active stimulation device (the electrodes) need to be implanted close enough to the genioglossus nerve branch 106 for effective stimulation, the neurostimulator implant needs to have the required electronics implanted close enough to the skin surface to send/receive wireless communications, the neurostimulator implant needs to be oriented such that it can receive wireless power. The neurostimulator implants, delivery devices, and implant procedures set out herein address these challenges and provide an implantable system for treating sleep apnea that includes stimulation of the genioglossus nerve branch 106 within the genioglossus muscle 112.

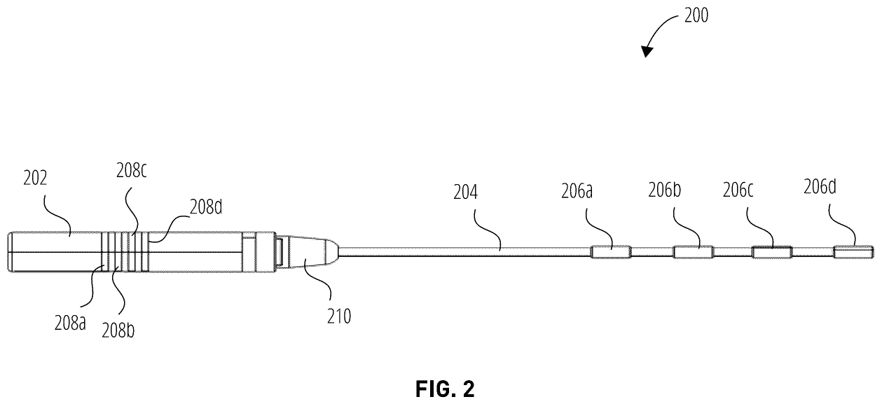

FIG. 2 illustrates an example neurostimulator implant 200 having a housing portion 202 and an electrode lead 204 extending from the housing portion 202. The housing portion 202 may house an electronics assembly of the neurostimulator implant 200. The electronics assembly within the housing portion 202 may include a wireless power receiver (e.g., an antenna), and a signal generator. Additionally, the electronics assembly may include a wireless communicators receiver/transmitter. Additionally, the electronics assembly may include sensor electronics. In examples, the electronics assembly may include a printed circuit board (PCB). In examples, the housing portion 202 is hermetically sealed. In examples, the neurostimulator implant 200 is battery-less.

The electrode lead 204 extends from the housing portion 202 and is flexible. The electrode lead 204 is joined to the housing portion 202 by a lead connector 210. The electrode lead 204 includes at least one electrode 206a, in this example four electrodes 206a, 206b, 206c, 206d. In other examples, the electrode lead 204 may comprise one, two, three, five, six, or more electrodes. The electrodes 206a, 206b, 206c, 206d are spaced from each other at a distal end of the electrode lead 204. The electrodes 206a, 206b, 206c, 206d are connected to the electronics within the housing portion 202. The electrodes 206a, 206b, 206c, 206d are metal, preferably a platinum-iridium alloy or titanium.

In examples, the electrodes 206a, 206b, 206c, 206d are operated by the electronics assembly housed in the housing portion 202 to provide electrical stimulation to stimulate a nerve of the patient. In other examples, the electrodes 206a, 206b, 206c, 206d are operated, by the electronics assembly housed in the housing portion 202, to sense or detect neural signals. In other examples, the electrodes 206a, 206b, 206c, 206d are operated, by the electronics assembly housed in the housing portion 202, to detect other (non-neural) parameters of the patient, for example bioimpedance, blood pressure, or heart rate. In examples, at least two of the electrodes 206a, 206b, 206c, 206d may comprise different materials, in particular different metals, and may be operated to detect a differential bioimpedance. In examples, the electrodes 206a, 206b, 206c, 206d are operable in a first mode to detect neural signals, and operable in a second mode to stimulate a nerve of the patient. In examples, some of the electrodes 206a, 206b, 206c, 206d are operable to detect neural signals, and others are operable to stimulate the nerve. In examples, some of the electrodes 206a, 206b, 206c, 206d are operable to detect/stimulate a nerve, and others are operable to sense other (non-neural) parameters. In examples, the neurostimulator implant 200 is implantable to sense and/or stimulate the hypoglossal nerve 108 shown in FIG. 1, in particular the genioglossus nerve branch 106 within the genioglossus muscle 112. In other examples, the neurostimulator implant 200 may be implantable to sense and/or stimulate the phrenic nerve and/or the ansa cervicalis nerve. Sensing and stimulation of the hypoglossal nerve and/or the phrenic nerve and/or the ansa cervicalis nerve may provide treatment for sleep apnea. In other examples, the neurostimulator implant 200 may be implantable to sense and/or stimulate other nerves, including the greater occipital nerve, the tibial nerve, the sacral nerve (e.g., to treat urinary incontinence), or the vagus nerve (e.g., to regulate pancreatic secretion).

In examples, in operation the electrodes 206a, 206b, 206c, 206d of the neurostimulator implant 200 are provided with an electrical signal, such as a current, to stimulate the nerve. In examples, the electrical signal may be a voltage-regulated stimulation.

In other examples, the neurostimulator implant 200 may additionally be operable as a diagnostic implant, for example a neurodiagnostic implant, operable to detect one or more neural signals in a nerve. In such examples the electrodes 206a, 206b, 206c, 206d are operable to detect neural signals. The neural signals may be analysed for the purposes of detecting, monitoring and/or diagnosing a condition.

In other examples, the neurostimulator implant 200 may additionally be operable as a diagnostic implant operable to detect one or more physiological parameters, for example body temperature, heart rate, electromyography (EMG), electrocardiogram (ECG), respiration rate, blood pressure, oxygen saturation, and/or blood gas concentration (e.g., oxygen, carbon dioxide, carbon monoxide).

In examples, the housing portion 202 may have an outer diameter of less than about 5 millimetres, for example between about 1 millimetre and about 5 millimetres, for example between about 1 millimetre and about 3 millimetres, for example about 3 millimetres. The housing portion 202 may have a length of up to about 30 millimetres, for example up to about 25 millimetres, for example up to about 20 millimetres, for example about 20 millimetres. In examples, the electrode lead 204 may have a diameter of between about 0.3 millimetres to about 1.5 millimetres, for example between about 0.5 millimetres and 1.3 millimetres, for example about 0.8 millimetres. The electrode lead 204 may have a length of up to about 200 millimetres, for example up to about 150 millimetres, for example up to about 120 millimetres, for example about 120 millimetres. The electrodes 206a, 206b, 206c, 206d may each have a length (along the electrode lead 204) of up to about 15 millimetres, for example up to about 10 millimetres, for example up to about 5 millimetres, for example about 3 millimetres. The electrodes 206a, 206b, 206c, 206d may be spaced from each other along the electrode lead 204 by at least 2 millimetres, for example at least 3 millimetres, for example at least 5 millimetres.

However, it will be appreciated that the dimensions of the housing portion 202 would correspond to the size of the electronics assembly within the housing portion 202, and the length of the electrode lead 204 would correspond to the anatomy surrounding the targeted nerve, so a shorter or longer electrode lead 204 may be appropriate depending on the depth of the nerve within the patient's tissue.

As also shown in FIG. 2, the housing portion 202 further includes one or more ring electrodes 208a, 208b, 208c, 208d. The ring electrodes 208a, 208b, 208c, 208d are spaced from each other and extend at least partly circumferentially about the housing portion 202. In this example the housing portion 202 comprises four ring electrodes 208a, 208b, 208c, 208d. In other examples, the housing portion 202 may comprise one, two, three, five, six, or more ring electrodes. The ring electrodes 208a, 208b, 208c, 208d are connected to the electronics within the housing portion 202, for example via a feedthrough. The ring electrodes 208a, 208b, 208c, 208d are metal, preferably a platinum-iridium alloy or titanium.

In examples, the ring electrodes 208a, 208b, 208c, 208d are operable to sense and/or stimulate a nerve of the patient in the same manner as the electrodes 206a, 206b, 206c, 206d described above. In examples, the ring electrodes 208a, 208b, 208c, 208d are operable to detect one or more physiological parameters, for example body temperature, heart rate, electromyography (EMG), electrocardiogram (ECG), respiration rate, blood pressure, oxygen saturation, and/or blood gas concentration (e.g., oxygen, carbon dioxide, carbon monoxide).

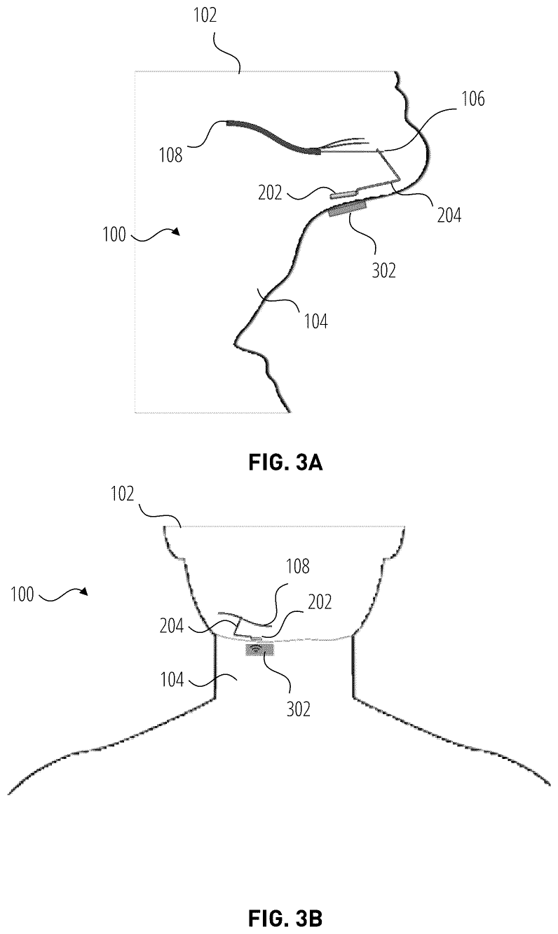

FIG. 3A and FIG. 3B illustrate a first example implantation of the neurostimulator implant 200 of FIG. 2 in the patient 100 illustrated in FIG. 1. As shown in FIG. 3A, the neurostimulator implant 200 is implanted under the chin of the patient 100. The electrode lead 204 extends proximal to the genioglossus nerve branch 106 of a hypoglossal nerve 108 within the genioglossus muscle 112, in this example the genioglossus nerve branch 106 of the right hypoglossal nerve 108. The housing portion 202 is implanted in subcutaneous tissue in the neck 104. As described further below, the housing portion 202 is implanted to be substantially parallel with the skin of the neck 104. An external device 302 is attached to the neck 104 in alignment with the housing portion 202. The external device 302 may provide wireless power transfer to the neurostimulator implant 200 and/or wirelessly communicate with the neurostimulator implant 200. The external device 302 may be operable to control the neurostimulator implant 200. In preferred examples the neurostimulator implant 200 is battery-less, and the external device 302 provides power for operating the neurostimulator implant 200.

The neurostimulator implant 200 illustrated in FIG. 3A and FIG. 3B may be used to detect and/or treat sleep apnea. For example, the neurostimulator implant 200 may be operable to detect one of more indicators of sleep apnea, such as heart rate, respiratory rate, mandibular movement(s), thoracic movement(s), diaphragm movement(s), and/or Electromyograph, EMG. In particular, one or more of the electrodes 206a, 206b, 206c, 206d and/or one or more of the ring electrodes 208a, 208b, 208c, 208d may be operable to detect one or more indicators of sleep apnea. In examples, the neurostimulator implant 200 may be operable to stimulate the genioglossus nerve branch 106 of the right hypoglossal nerve 108 within the genioglossus muscle 112. In particular, one or more of the electrodes 206a, 206b, 206c, 206d may be operable to stimulate the genioglossus nerve branch 106 of the right hypoglossal nerve 108 within the genioglossus muscle 112. Such stimulation causes innervation of the genioglossus muscle 112 to pull the tongue forward (anteriorly) to treat obstructive sleep apnea (OSA). The stimulation may directly innervate the genioglossus muscle 112 (particularly the parts of the genioglossus muscle 112 proximal to the electrode lead 204), and/or may stimulate the genioglossus nerve branch 106 to innervate the genioglossus muscle 112.

It will be appreciate that the neurostimulator implant 200 may alternatively be implanted at the other hypoglossal nerve (the left hypoglossal nerve). Stimulation of a single hypoglossal nerve, for example the right or left hypoglossal nerve 108, may be termed unilateral hypoglossal stimulation.

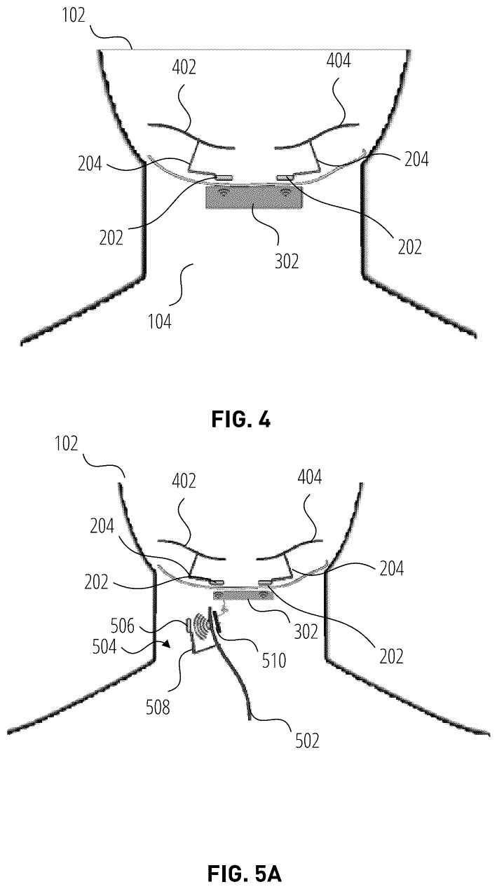

FIG. 4 illustrates a further example implantation of the neurostimulator implant 200 of FIG. 2 in the patient 100 illustrated in FIG. 1. In this example, a first neurostimulator implant 200 is implanted at the right hypoglossal nerve 402 in the same manner as described with reference to FIG. 3A and FIG. 3B, and a second neurostimulator implant 200 is implanted at the left hypoglossal nerve 404. The neurostimulator implant 200 implanted at the left hypoglossal nerve 404 is implanted in the same manner as described with reference to FIG. 3A and FIG. 3B, but the electrode lead 204 is implanted proximal to the left hypoglossal nerve 404.

As shown, a single external device 302 may be provided to power and/or communicate with both of the neurostimulator implants 200. Alternatively, different external devices 302 may be provided for the two neurostimulator implants 200. In some examples, the two neurostimulator implants 200 may be in direct wireless communication with each other, or they may both wirelessly communicate with the external device 302.

The neurostimulator implants 200 are operated in the same manner as described with reference to FIG. 3A and FIG. 3B, for example to detect one of more indicators of sleep apnea, and/or stimulate the right hypoglossal nerve 402 and the left hypoglossal nerve 404 and/or the genioglossus muscle 112. Such stimulation may cause innervation of the genioglossus muscle 112 to pull the tongue forward (anteriorly) to alleviate obstructive sleep apnea (OSA). The stimulation may directly innervate the genioglossus muscle 112 (particularly the parts of the genioglossus muscle 112 proximal to the electrode lead 204), and/or may stimulate the genioglossus nerve branch 106 to innervate the genioglossus muscle 112.

Stimulation of both the left hypoglossal nerve 404 and the right hypoglossal nerve 402 may be termed bilateral hypoglossal stimulation.

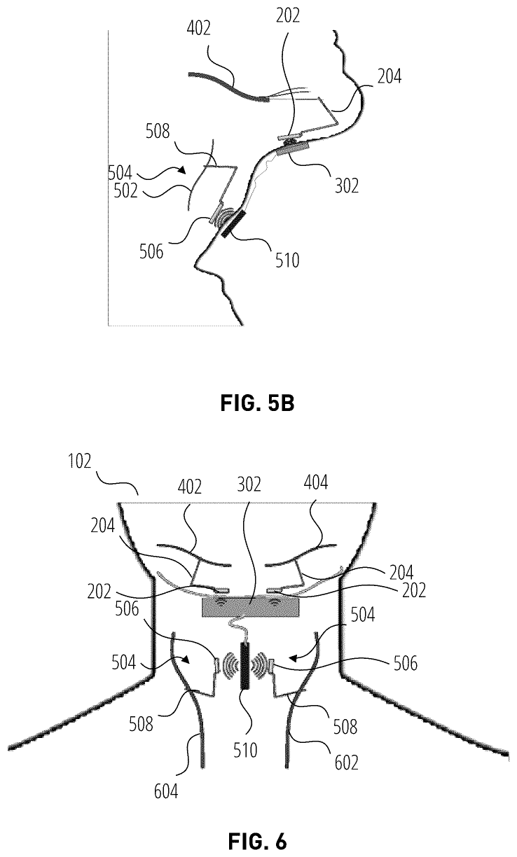

FIG. 5A and FIG. 5B illustrate a further example implantation of the neurostimulator implant 200 of FIG. 2 in the patient 100 illustrated in FIG. 1. In this example, two neurostimulator implants 200 are implanted at the right hypoglossal nerve 402 and the left hypoglossal nerve 404, respectively, as described with reference to FIG. 4. Optionally, one a single neurostimulator implant 200 may be provided, at the right hypoglossal nerve 402 or at the left hypoglossal nerve 404. A third neurostimulator implant 504 is implanted in the patient's neck with the electrode lead 508 being implanted proximal to the phrenic nerve 502, in particular the right phrenic nerve 502. The neurostimulator implant 504 may be implanted to stimulate the phrenic nerve 502 within the ansa cervicalis, or at a position where the phrenic nerve 502 has branched from the ansa cervicalis. The neurostimulator implant 504 is the same as the neurostimulator implant 200 described with reference to FIG. 2 and is implanted to sense and/or stimulate the phrenic nerve 502. The phrenic nerve 502 innervates the diaphragm and stimulation of the phrenic nerve 502 may trigger inhalation, helping to alleviate central sleep apnea (CSA) by improving regulation of patient breathing. Stimulating the phrenic nerve 502 in addition to, or instead of, the right and left hypoglossal nerve 402, 404 may provide treatment for combined obstructive sleep apnea (OSA) and central sleep apnea (CSA), or one or other of obstructive of sleep apnea (OSA) and central sleep apnea (CSA).

As shown in FIG. 5A and FIG. 5B, the housing portion 506 of the neurostimulator implant 504 may be implanted in subcutaneous tissue and an external device 510 may be attached to the patient's skin in alignment with the housing portion 506 to provide wireless power and/or communications for the neurostimulator implant 504. In examples, the external device 510 may be separate to the external device 302, or it may be an antenna of the external device 302. In other examples, the external device 302 may directly power and/or communicate with the neurostimulator implant 504. In examples, each of the neurostimulator implants 200 and the neurostimulator implant 504 may be wireless communication with each other, or they may all wirelessly communicate with the or each external device 302.

It will be appreciate that the neurostimulator implant 504 may alternatively be implanted at the other phrenic nerve (the right phrenic nerve). Stimulation of a single phrenic nerve, for example the right or left phrenic nerve, may be termed unilateral phrenic stimulation.

FIG. 6 illustrates a further example in which a second neurostimulator implant 504 is implanted at the left phrenic nerve 602 in addition to the first neurostimulator implant 504 implanted at the right phrenic nerve 604 and the two neurostimulator implants 200 implanted at the right hypoglossal nerve 402 and the left hypoglossal nerve 404, respectively. The second neurostimulator implant 504 may be implanted to stimulate the left phrenic nerve 602 and the right phrenic nerve 604 within the ansa cervicalis, or at a position where the left phrenic nerve 602 and right phrenic nerve 604 have branched from the ansa cervicalis.

The external device 510 and/or external device 302 may power and/or communicate with the second neurostimulator implant 504 as well as the first.

Stimulating the left phrenic nerve 602 and/or right phrenic nerve 604, in addition to, or instead of, the hypoglossal nerve 402, 404 may provide treatment for combined obstructive sleep apnea (OSA) and central sleep apnea (CSA), or one or other of obstructive of sleep apnea (OSA) and central sleep apnea (CSA).

Stimulation of both the left phrenic nerve 602 and the right phrenic nerve 604 may be termed bilateral phrenic stimulation.

In some examples, the first and/or second neurostimulator implants 504 may be implanted to stimulate the ansa cervicalis nerve, which is close to the phrenic nerve 502.

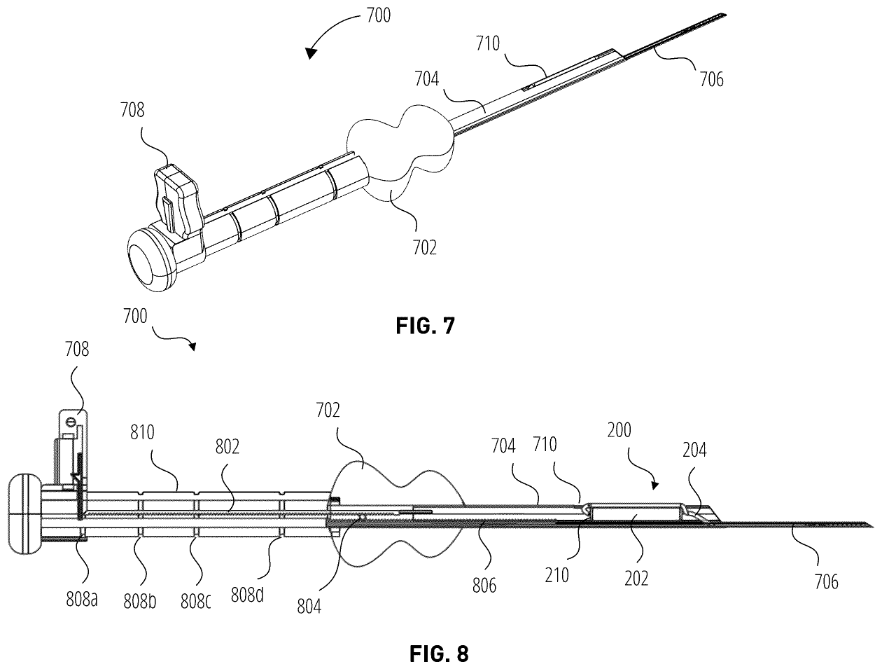

FIG. 7 illustrates a delivery device 700 for implanting the neurostimulator implant 200 of FIG. 2 at the hypoglossal nerve 108 as shown in any of FIG. 3A to FIG. 6. The delivery device 700 may additionally be used to implant the neurostimulator implant 200 at the phrenic nerve 502 and/or ansa cervicalis nerve as shown in FIG. 5A to FIG. 6.

As shown in FIG. 7, the delivery device 700 comprises a handle 702 and a first needle 704 fixed to and extending from the handle 702. A second needle 706 extends from the first needle 704 in a direction away from the handle 702. The second needle 706 is retractable relative to the first needle 704 by operating the control slider 708 as described further below. The first needle 704 is larger (has a lower gauge) than the second needle 706. In examples, the first needle 704 may have a gauge of between 6 gauge and 15 gauge, for example 10 gauge. In examples, the second needle 706 may have a gauge of between 15 gauge and 25 gauge, for example 20 gauge.

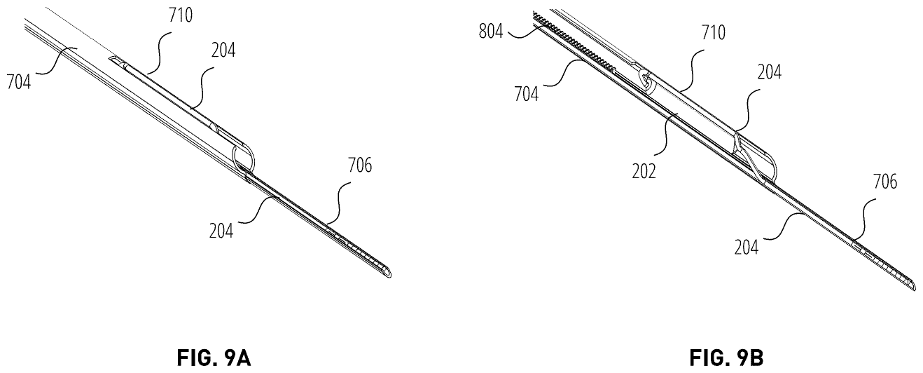

FIG. 8 shows a cross-section through the delivery device 700 of FIG. 7, and FIG. 9A and FIG. 9B show details of the first needle 704 and the second needle 706. The second needle 706 is slidably received in the first needle 704 and extends beyond the first needle 704 away from the handle 702. The second needle 706 is retractable into the first needle 704 during operation, as described in detail below. The control slider 708 is operable to retract and extend the second needle 706 relative to the first needle 704 and the handle 702. The control slider 708 is operably coupled to the second needle 706 by a rack and pinion mechanism. The rack and pinion mechanism includes a first rack portion 802 joined to the control slider 708 and extending into the handle 702. A second rack portion 806 is joined to the second needle 706 and extends into the handle 702. A pinion 804 is rotatably mounted in the handle 702 and acts between the first rack portion 802 and the second rack portion 806. Accordingly, moving the control slider 708 towards the handle 702 will cause the second rack portion 806 and the second needle 706 to move towards the handle 702—i.e., to retract the second needle 706 into the first needle 704.

As shown in FIG. 8, the handle 702 includes a guide portion 810 extending from the handle 702 in a direction opposite to the first needle 704. The control slider 708 can slide along the guide portion 810. The guide portion 810 includes one or more, in this example four, stop positions 808a, 808b, 808c, 808d that provide pre-defined operational positions of the control slider 708. The stop positions 808a, 808b, 808c, 808d may comprise a hole or groove and the control slider 708 may include a ball detent for engaging the holes or grooves to stop the control slider 708 at the stop positions 808a, 808b, 808c, 808d. Accordingly, the control slider 708 can be moved along the guide portion 810 between the different stop positions 808a, 808b, 808c, 808d to move the second needle 706 between different predefined positions during use of the delivery device 700, described in detail below.

As shown in FIG. 8, FIG. 9A and FIG. 9B, the neurostimulator implant 200 illustrated in FIG. 2 is received in the delivery device 700. In particular, the housing portion 202 is received in the first needle 704 and the electrode lead 204 is received in the second needle 706. During use, as described further below, retraction of the second needle 706 implants the electrode lead 204 in the patient, after which the housing portion 202 can be ejected from the first needle 704 in a two-stage implantation process.

In this example, the housing portion 202 is oriented such that the lead connector 210 is directed towards the handle 702. The electrode lead 204 extends from the lead connector 210 and overlies the housing portion 202 before entering the second needle 706. As illustrated in FIG. 7 and FIG. 8, the first needle 704 includes a slot 710 to accommodate the part of the electrode lead 204 that overlies the housing portion 202 within the first needle 704. The slot 710 advantageously permits the housing portion 202 to be implanted at a different angle to the electrode lead 204 without applying undue bending stress to the electrode lead 204, as described below.

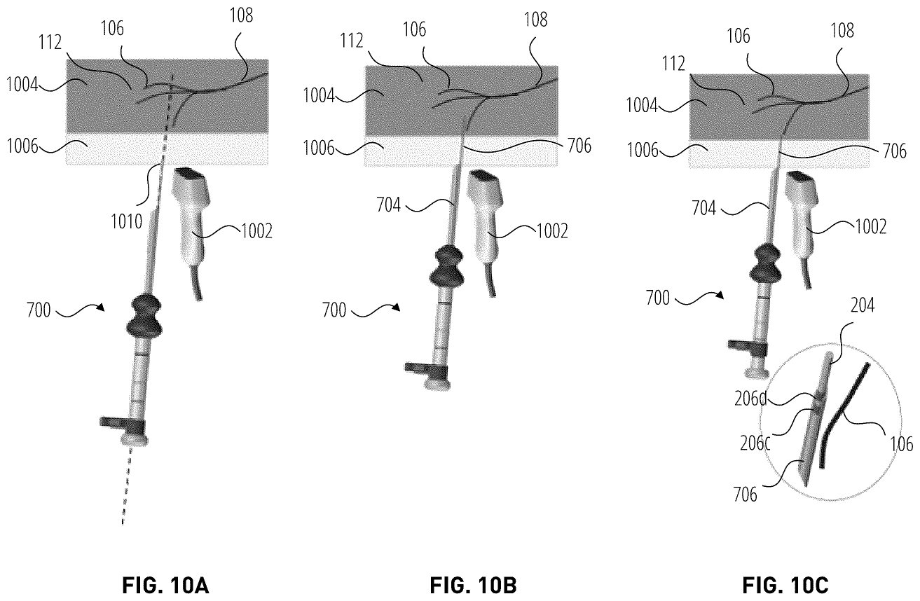

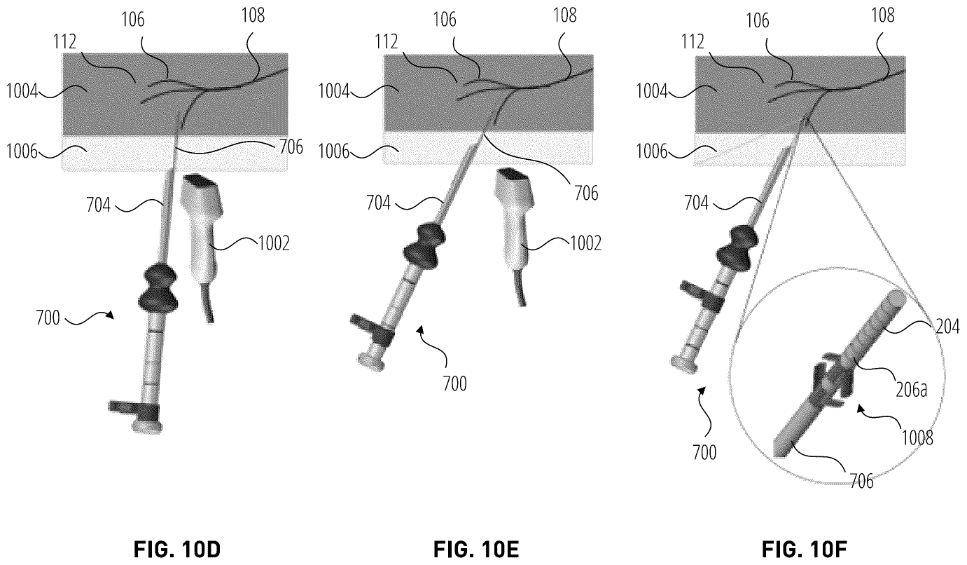

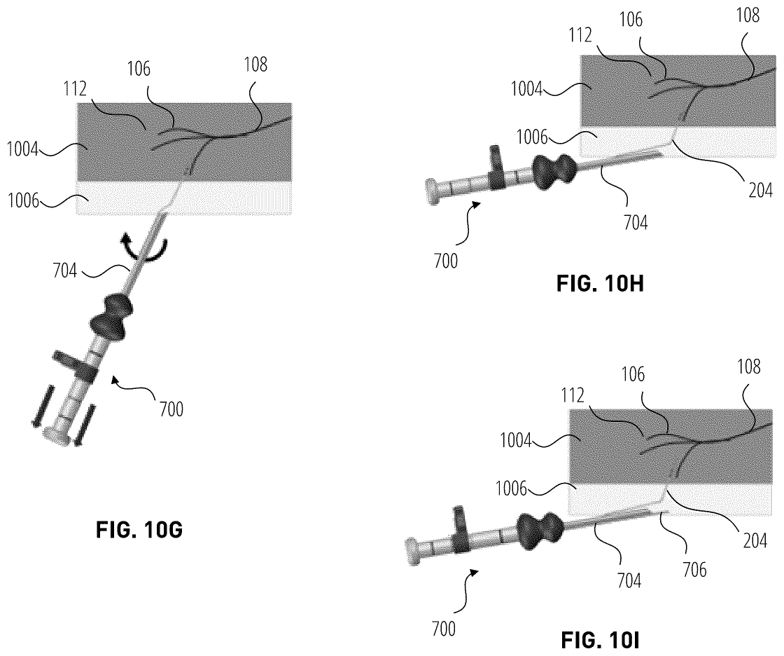

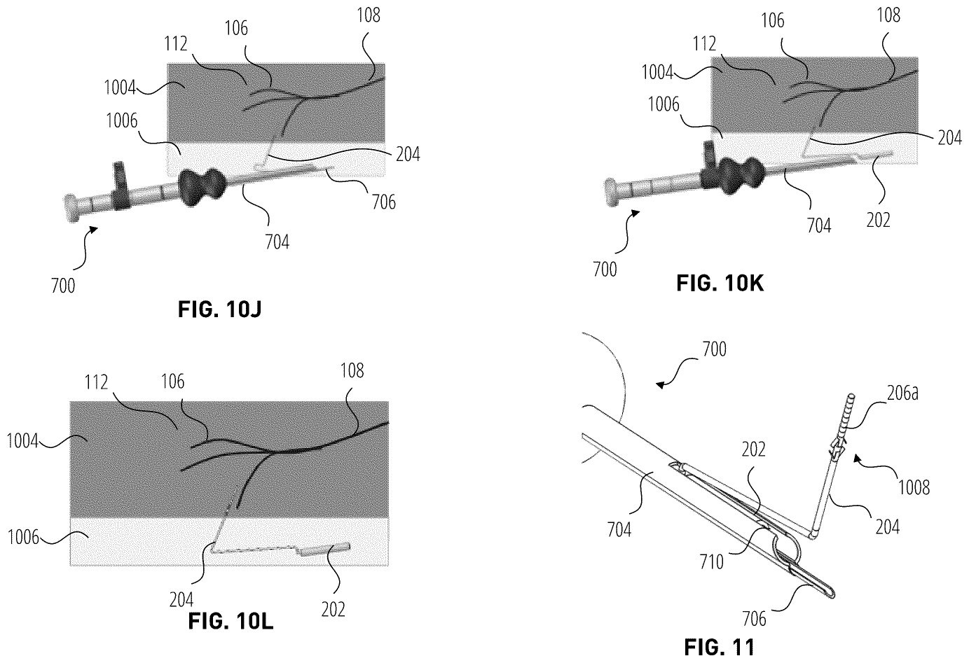

FIG. 10A to FIG. 10L illustrate a method of implanting the neurostimulator implant 200 in a patient using the delivery device 700 described with reference to FIG. 7 to FIG. 9B. In this example, the neurostimulator implant 200 is implanted to target the genioglossus nerve branch 106 of the Hypoglossal nerve 108 within the genioglossus muscle 112, as shown in FIG. 1.

As shown in FIG. 10A, initially the neurostimulator implant 200 is provided in the delivery device 700 in the manner described with reference to FIG. 7 to FIG. 9B. In particular, the second needle 706 is in an extended position. An ultrasound device 1002 is used to guide the second needle 706 towards the genioglossus nerve branch 106 of the Hypoglossal nerve 108 during implantation.

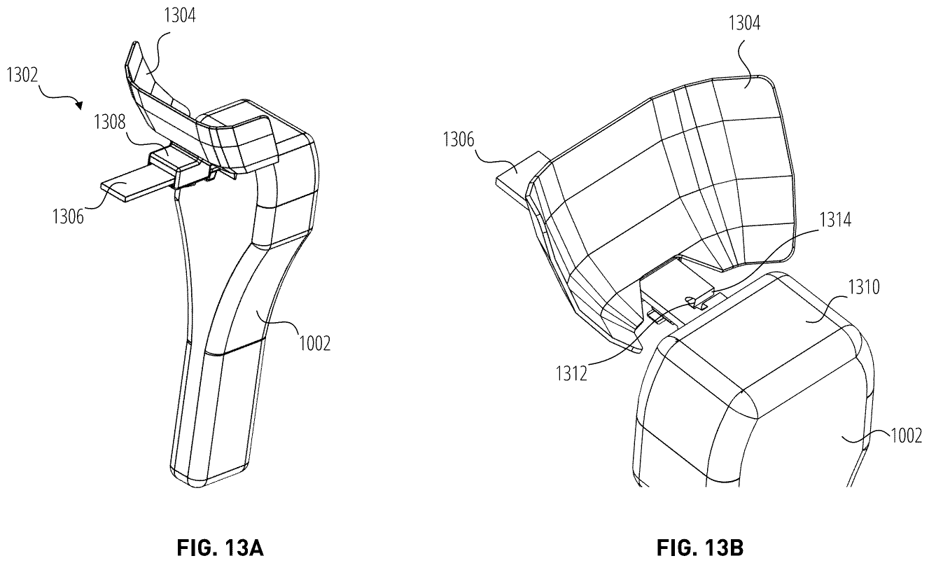

The second needle 706 enters the patient's tissue at an insertion point 1010. The insertion point 1010 is located in the following manner: with the patient's head tilted back and the neck in a hyperextended state, the operator identifies the posterior edge of the mandibular symphysis at the midline. This point is the insertion point 1010 for the second needle 706. As described further with reference to FIG. 13A to FIG. 15B, the insertion point 1010 may be located using a guide 1302 and/or a scan, in particular an ultrasound or X-ray.

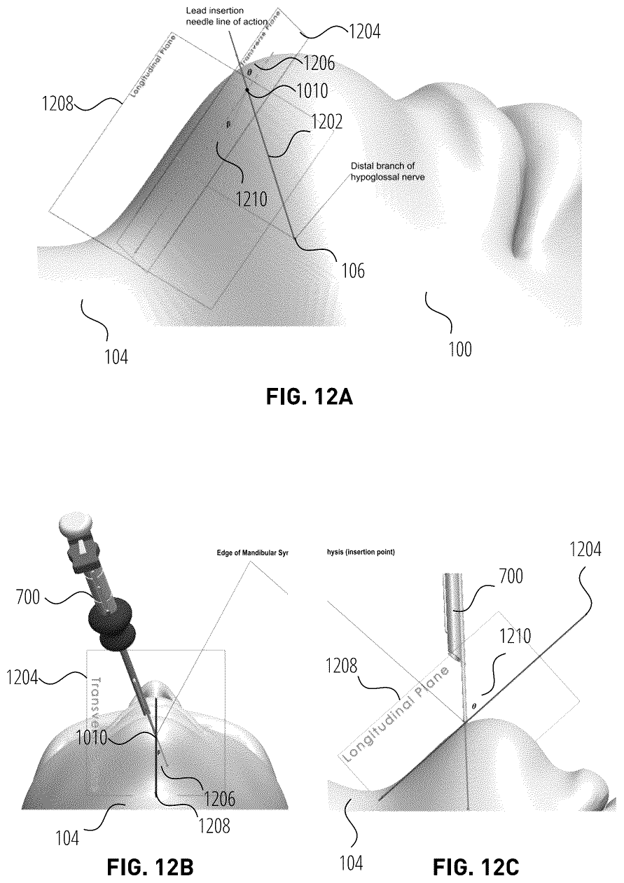

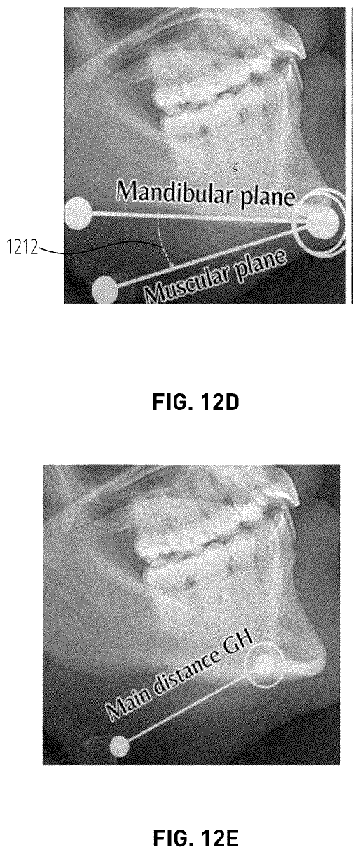

The second needle 706 is inserted at the insertion point 1010 with a predefined insertion orientation relative to the patient. In particular, the insertion orientation is defined by two angles: θ and β. The insertion orientation is determined based on the method described in more detail with reference to FIG. 12A to FIG. 12C.

Once the insertion point 1010 and insertion orientation have been determined, the second needle 706 is pushed into the patient's tissue, through the skin 1006 and into the subcutaneous tissue 1004, as shown in FIG. 10B. The second needle 706 is pushed towards the genioglossus muscle 112 and specifically the genioglossus nerve branch 106 of the Hypoglossal nerve 108. The ultrasound device 1002 provides an image of the position of the second needle 706 and can be used to guide the second needle 706 into proximity with the genioglossus nerve branch 106.

As shown in FIG. 10C, the position of the second needle 706, and thus the position of the electrode lead 204, can be tested by partially retracting the second needle 706 to expose at least some of the electrodes 206a, 206b, 206c, 206d on the electrode lead 204. In this example, as shown in the magnified detail, the second needle 706 is retracted to expose electrodes 206c and 206d. Referring to FIG. 8, the second needle 706 can be retracted to this position by moving the control slider 708 from the first stop position 808a to the second stop position 808b.

In the position shown in FIG. 10C the position of the electrode lead 204 can be tested by powering the neurostimulator implant 200. The neurostimulator implant 200 may be powered by a power system provided in the delivery device 700, or by an external device. The neurostimulator implant 200 can be powered and controlled to provide sensing or stimulation to determine if the electrode lead 204 is properly positioned with respect to the genioglossus nerve branch 106. This testing may include monitoring a reaction of the patient to stimulation by the neurostimulator implant 200.

As shown in FIG. 10D, if the position of the electrode lead 204 is determined to be unacceptable, the second needle 706 can be re-extended, specifically by moving the control slider 708 back to the first stop position 808a. The delivery device 700 can then be manipulated to move the second needle 706 into a better position relative to the genioglossus nerve branch 106, for example as shown in FIG. 10E. Repositioning the second needle 706 may include rotating the delivery device 700 so that the second needle 706 is closer to parallel with the genioglossus nerve branch 106.

Preferably, the end of the electrode lead 204 with the electrodes 206a, 206b, 206c, 206d is approximately parallel with the genioglossus nerve branch 106 so that each of the electrodes 206a, 206b, 206c, 206d can be operated to sense and/or stimulate the genioglossus nerve branch 106. Repositioning the second needle 706 may also include percutaneously positioning the tip of the first needle 704 in the patient, for example in the skin 1006 or subcutaneous tissue 1004.

Once the second needle 706 is determined to be appropriately positioned relative to the genioglossus nerve branch 106 within the genioglossus muscle 112, as shown in FIG. 10E, the second needle 706 can be more fully retracted to implant the electrode lead 204. The second needle 706 can be fully retracted by moving the control slider 708 to the fourth stop position 808d as shown in FIG. 8.

Preferably, as shown in FIG. 10F, retraction of the second needle 706 deploys anti-migration tines 1008 to anchor the electrode lead 204 in position. In particular, the anti-migration tines 1008 may be sprung tines attached to the electrode lead 204 between the housing portion 202 and the electrodes 206a, 206b, 206c, 206e. Accordingly, the anti-migration tines 1008 are not released during the position testing described with reference to FIG. 10C. However, when the second needle 706 is more fully retracted the anti-migration tines 1008 are released from within the second needle 706 and deploy outwards to anchor the electrode lead 204 in position.

As illustrated in FIG. 10G, once the electrode lead 204 has been implanted and preferably anchored, the delivery device 700 is partially withdrawn from the patient such that the first needle 704 is removed from the subcutaneous tissue 1004 and skin 1006. The delivery device 700 can be simultaneously or subsequently rotated. The slack in the electrode lead 204, due to the arrangement of the electrode lead 204 in the second first needle 704 and the second needle 706, and the slot 710 in the first needle 704 (see FIG. 7 to FIG. 9B) permits the retraction and rotation of the delivery device 700 without applying pulling or bending stress to the electrode lead 204. Advantageously, this prevents dislodgement of the electrode lead 204 from the implanted position. In particular, as shown in FIG. 11, as the delivery device 700 is rotated the part of the electrode lead 204 initially overlying the housing portion 202 within the first needle 704 can move out of the first needle 704 through the slot 710.

As shown in FIG. 10G and FIG. 10H, the delivery device 700 is rotated so that the first needle 704 is closer to parallel with the skin 1006. Advantageously, this permits the housing portion 202 to be implanted at a different angle the electrode lead 204, particularly an angle closer to parallel with the skin 1006.

As shown in FIG. 10H, once the delivery device 700 has been rotated, the first needle 704 is percutaneously repositioned for implanting the housing portion 202.

As shown in FIG. 10I, preferably the second needle 706 is partially redeployed to assist in moving the first needle 704 through the patient's skin 1006 and subcutaneous tissue 1004. The second needle 706 can be partially redeployed by moving the control slider 708 to the third stop position 808c shown in FIG. 8.

As shown in FIG. 10J, the first needle 704 is pushed into the patient's skin 1006 and subcutaneous tissue 1004 to a position for implanting the housing portion 202. During this movement, the electrode lead 204 bends and passes through the slot 710 in the first needle 704, preventing dislodgement of the implanted end of the electrode lead 204.

FIG. 10K illustrates ejection of the housing portion 202 from the first needle 704. The housing portion 202 is ejected from the first needle 704 my moving the control slider 708 to a maximum position towards the handle 702. In this position, as most clear in FIG. 8, the end of the first rack portion 802 will push the housing portion 202 out of the end of the first needle 704, as shown in FIG. 10K. The operator may gradually withdraw the delivery device 700 from the patient's tissue as they push the control slider 708 to the maximum position so that the housing portion 202 is implanted in the space created by the first needle 704.

The delivery device 700 can then be removed from the patient and FIG. 10L shows the implanted neurostimulator implant 200. As shown, the end of the electrode lead 204 with the electrodes has been implanted and anchored proximal to the genioglossus nerve branch 106 within the genioglossus muscle 112. The housing portion 202 has been implanted in the skin 1006, and the electrode lead 204 is bent between the housing portion 202 and the electrodes.

Advantageously, the housing portion 202 is oriented approximately parallel to the skin 1006, which may be beneficial for wireless power transfer and/or wireless communications between the housing portion 202 and an external device. Additionally, this implantation position enables the neurostimulator implant 200 to target the genioglossus nerve branch 106 within the genioglossus muscle 112, where there is minimal tissue suitable for implanting the housing portion 202.

Accordingly, the method described with reference to FIG. 10A to FIG. 10L advantageously allows the neurostimulator implant 200 to target the genioglossus nerve branch 106 within the genioglossus muscle 112 using a minimally invasive procedure.

FIG. 12A to FIG. 12C illustrate the method of determining the insertion point 1010 and insertion orientation 1202 for the delivery device 700 during the implantation method described with reference to FIG. 10A to FIG. 10K. The method of determining the insertion point 1010 and the insertion orientation 1202 may be based on Benbassat et al (Benbassat B, Cambronne C, Gallini A, Chaynes P, Lauwers F, de Bonnecaze G. The specific branches leading to the genioglossus muscle: three-dimensional localisation using skin reference points. Surg Radiol Anat. 2020 May; 42(5):547-555. doi: Epub 2019 Dec. 9. PMID: 31820050.).

The insertion point 1010 is located at the posterior edge of the mandibular symphysis at the midline. The operator can find and mark this by feeling the mandibular symphysis through the patient's skin. An ultrasound and/or X-ray scan may provide additional information for locating the insertion point 1010.

As shown in FIG. 12A to FIG. 12C, the insertion orientation is defined by a first angle 1206 in a transverse plane 1204. The transverse plane 1204 is approximately the muscular plane of the patient's chin when the neck 104 is in a hyperextended position, which is also approximately the plane of the skin on the chin as shown in FIG. 12D. The first angle 1206 is positive or negative, depending on whether the left or right hypoglossal nerve is being targeted. The first angle 1206 is the angular offset from the longitudinal plane 1208 of the patient in the transverse plane 1204. As described below, the longitudinal plane 1208 is the median, or sagittal plane of the patient 100.

As shown in FIG. 12A to FIG. 12C, the insertion orientation is also defined by a second angle 1210 in a longitudinal plane 1208. The longitudinal plane 1208 is the patient's median (sagittal) plane and is perpendicular to the transverse plane 1204. The second angle 1210 is the angular offset from the transverse plane 1204 in the longitudinal plane 1208.

In examples, the first angle 1206 may be between about ±15 degrees and about ±30 degrees, for example between about ±18 degrees and about ±25 degrees, for example between about ±20 degrees and about ±23 degrees. In one particular example, the first angle 1206 may be about ±22 degrees, in particular about ±21.8 degrees.

In examples, the second angle 1210 may be between about 40 degrees and about 65 degrees, for example between about 45 degrees and about 55 degrees, for example between about 40 degrees and about 50 degrees. In particular example, the second angle 1210 is example between about 47 degrees and about 49 degrees, in particular about 48.1 degrees.

It will be appreciated however that the first angle 1206 and the second angle 1210 may be different for different patients, and the first angle 1206 and the second angle 1210 may be determined for a particular patient in advance of the implantation procedure, for example based on an ultrasound scan or other scan information.

In some examples, the insertion depth is also determined. The insertion depth is approximately the distance from the skin to the genioglossus nerve branch 106 within the genioglossus muscle 112. If the insertion depth is determined, it may be based on the below equations defined by Benbassat et al (Benbassat B, Cambronne C, Gallini A, Chaynes P, Lauwers F, de Bonnecaze G. The specific branches leading to the genioglossus muscle: three-dimensional localisation using skin reference points. Surg Radiol Anat. 2020 May; 42(5):547-555. doi: 10.1007/s00276-019-02390-w. Epub 2019 Dec. 9. PMID: 31820050.).

L=\sqrt{(Y)²+(0.6GH)²}

Y=\sqrt{(0.5GH)²+(0.2GH)²}

Where:

L is the insertion depth.

GH is a measured variable, defined as the distance from the posterior edge of the mandibular symphysis to the hyoid bone along the midline (in the transverse plane). The distance GH is shown in FIG. 12E.

In other examples, the insertion depth may be determined in advance of the implantation procedure based on a medical image, for example an X-ray or ultrasound scan. In other examples, the operator may use a scanner, in particular an ultrasound scanner, to monitor the insertion depth and position. In other examples, the insertion depth is not determined, and instead the operator may insert the delivery device 700 to a predetermined depth. In other examples, the insertion depth may be tested and/or verified by generating stimulation signals and testing a reaction of the patient to the stimulation signal, and/or by using bioimpedance sensing to detect when the electrodes are implanted close to the nerve.

As shown in FIG. 12D and FIG. 12E, to assist in determining the appropriate insertion orientation for a particular patient, a scan, in this case a cephalic radiograph, may be taken before the implantation procedure. As illustrated, the radiograph can be used to determine the angle 1212 between the mandibular plane and the muscular plane on the patient's chin, allowing proper localisation of the transverse plane 1204 as described above. In addition, if needed, the variable GH can be measured from the radiograph as shown in FIG. 12E.

FIG. 13A to FIG. 15B illustrate a first example guide 1302 for use in the implantation method described with reference to FIG. 10A to FIG. 10L. As shown in FIG. 13A and FIG. 13B, the guide 1302 includes a locating portion 1304 shaped to conform with the patient's chin for positioning the guide 1302 relative to the patient. The locating portion 1304 is mounted on a rail 1306 via a slider 1308, and the ultrasound device 1002 is also attached to the rail 1306. Accordingly, the position of the locating portion 1304 is adjustable relative to the ultrasound device 1002. In this way, the ultrasound sensor window 1310 of ultrasound device 1002 is positioned appropriately relative to the patient during implantation.

As illustrated in FIG. 13B, the rail 1306 includes a guide channel 1312 for the second needle 706 of the delivery device 700 illustrated in FIG. 7. The guide channel 1312 is sized and shaped to define the insertion point and insertion orientation of the second needle 706 for implanting the neurostimulator implant 200 at the genioglossus nerve branch 106 of the hypoglossal nerve 108 as described with reference to FIG. 10A to FIG. 10F. The guide channel 1312 includes a slot 1314 for removal of the guide 1302 before the delivery device 700 is rotated as described with reference to FIG. 10G. Accordingly, the guide 1302 is used to guide the second needle 706 to the insertion point 1010 (see FIG. 10A) and to maintain the insertion orientation until the second needle 706 is appropriately positioned relative to the genioglossus nerve branch 106.

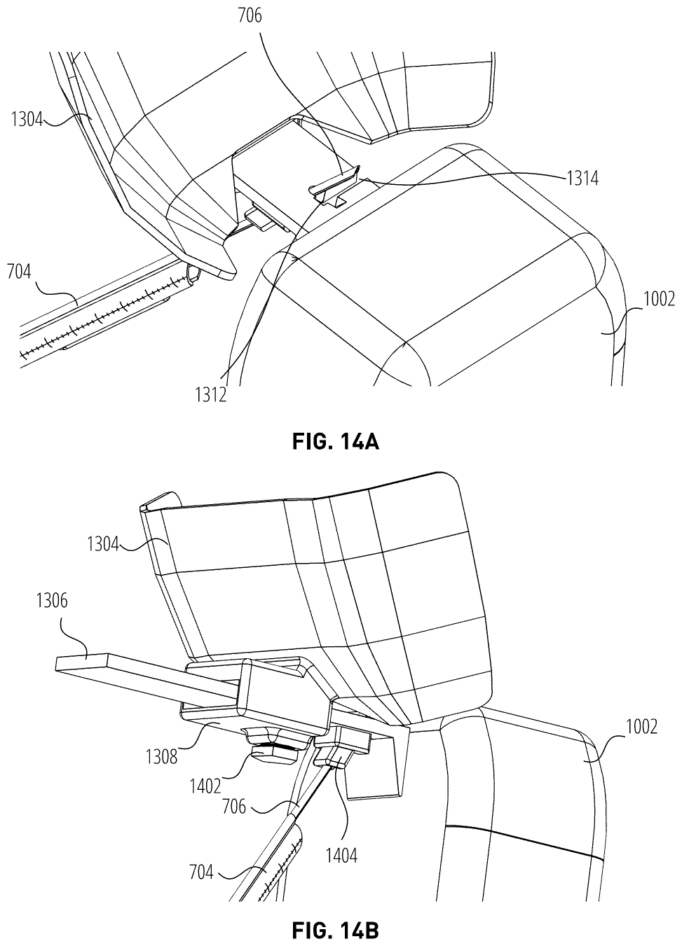

The rail 1306 can be moved, based on the images obtained from the ultrasound device 1002, to align the guide channel 1312 with the insertion point. As shown in FIG. 14A and FIG. 14B, the slider 1308 includes a lock 1402 for fixing the position of the locating portion 1304 along the rail 1306 once the guide channel 1312 has been aligned with the insertion point. Accordingly, the guide 1302 is adjustable for the particular patient.

The rail 1306 includes a guide channel portion 1404 through which the guide channel 1312 extends. The guide channel 1312 thereby defines the insertion orientation for the second needle 706 as described above.

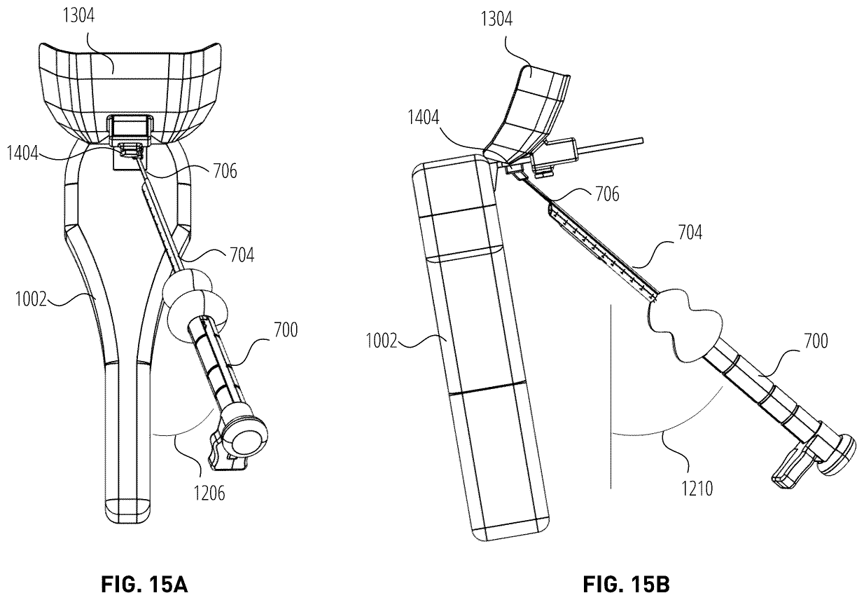

In particular, as shown in FIG. 15A and FIG. 15B, the insertion orientation is defined by a first angle 1206 and a second angle 1210 determined based on the method described with reference to FIG. 12A to FIG. 12C.

As per the method described with reference to FIG. 12A to FIG. 12C, the first angle 1206 may be between about ±15 degrees and about ±30 degrees, for example between about ±18 degrees and about ±25 degrees, for example between about ±20 degrees and about ±23 degrees. In one particular example, the first angle 1206 may be about ±22 degrees, in particular about ±21.8 degrees. In examples, the guide 1302 is adjustable so that the first angle 1206 defined by the guide channel portion 1404 can be adjusted, for example adjusted within the above ranges.

In examples, the second angle 1210 may be between about 40 degrees and about 60 degrees, for example between about 45 degrees and about 55 degrees, for example between about 40 degrees and about 50 degrees. In particular example, the second angle 1210 is example between about 47 degrees and about 49 degrees, in particular about 48.1 degrees. In examples, the guide 1302 is adjustable so that the second angle 1210 defined by the guide channel portion 1404 can be adjusted, for example adjusted within the above ranges.

Before use, a scan (e.g., an X-ray such as that shown in FIG. 12D and FIG. 12E) may be taken to determine an appropriate first angle 1206 and second angle 1210.

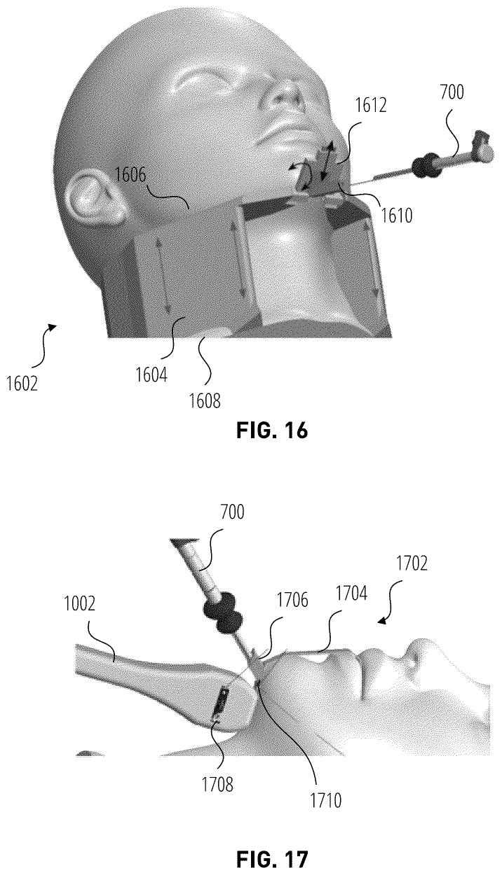

FIG. 16 to FIG. 18 illustrate further examples of guides 1602, 1702, 1802.

In the example of FIG. 16 the guide 1602 comprises a brace 1604 that is positionable between the mandible 1606 and the neck/shoulder 1608 of the patient. A guide channel portion 1610 with a guide channel is mounted to the brace 1604 via a mounting portion 1612. The mounting portion 1612 is movably mounted to the brace 1604 so that the position and orientation of the guide channel portion 1610 is adjustable.

Advantageously, the brace 1604 locates against the mandible 1606 of the patient and so variations between the mandibular plane and muscular plane (see FIG. 12D) are less significant. In addition, the guide 1602 permits the operator to fully adjust the position and orientation of the guide channel portion 1610 and therefore also the position and orientation of the delivery device 700 to provide the best position and orientation for insertion of the delivery device 700. The brace 1604 also advantageously acts to restrict movement of the patient during implantation.

In the example of FIG. 17, the guide 1702 includes a mouthpiece 1704. The mouthpiece 1704 is inserted into the patient's mouth and clamped there by the patient biting on the mouthpiece 1704. The mouthpiece 1704 extends over the chin of the patient and comprises a guide channel portion 1706 located approximately at the rear edge of the mandibular symphysis. The guide channel portion 1706 may be movably mounted, for example rotatably mounted, to the mouthpiece 1704 to provide adjustability of the position and orientation of the guide channel portion 1706. An ultrasound device 1002 is also mounted to the guide 1702 in a similar manner to as described above. The ultrasound device 1002 is attached to the guide 1702 so that it is directed towards the patient for imaging the implant location during use.

In this example, a first inclinometer 1708 is provided on the ultrasound device 1002. The first inclinometer 1708 measures an inclination of the ultrasound device 1002. During use, based on the ultrasound images obtained by the ultrasound device 1002, the ultrasound device 1002 is moved so that it is approximately parallel to the transverse plane (1204, see FIG. 12A to FIG. 12C). A second inclinometer 1710 is provided on the guide channel portion 1706. Accordingly, the angle difference between the angular measurements at that first inclinometer 1708 and the insertion point 1010 gives the angular offset between the transverse plane and the guide channel portion 1706. In this way, the guide channel portion 1706 can be appropriately positioned and oriented for implantation.

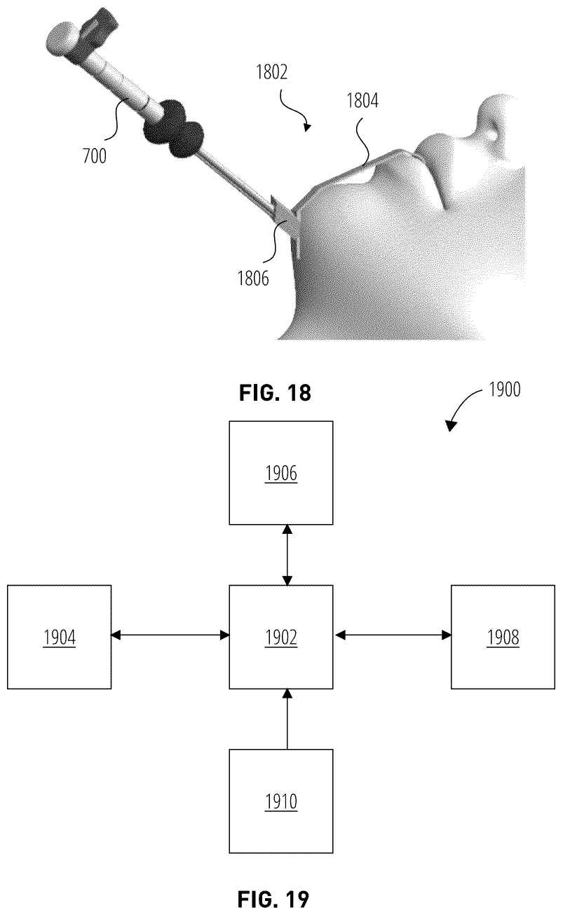

In the example of FIG. 18, the guide 1802 includes a mouthpiece 1804. The mouthpiece 1804 is inserted into the patient's mouth and clamped there by the patient biting on the mouthpiece 1804. The mouthpiece 1804 extends over the chin of the patient and comprises a guide channel portion 1806 located approximately at the rear edge of the mandibular symphysis. In this example the guide channel portion 1806 is fixedly mounted to the mouthpiece 1804 and this guide 1802 does not provide for adjustability of the insertion position and orientation. However, the guide 1802 may be configured for a particular patient, for example by 3D printing based on an earlier scan. Or, a number of guides 1802 with different sizes and dimensions may be provided and the most appropriate guide 1802 can be selected based on a scan.

FIG. 19 illustrates a neurostimulator implant system 1900 that includes a hypoglossus neurostimulator implant 1904, a phrenic nerve neurostimulator implant 1906, and a continuous positive air pressure device, CPAP device 1908. The hypoglossus neurostimulator implant 1904 may comprise one or two neurostimulator implants 200 as described with reference to FIG. 3A to FIG. 4. The phrenic nerve neurostimulator implant 1906 may include one or two neurostimulator implants 504 as described with reference to FIG. 5A to FIG. 6, and may stimulate the phrenic nerve and/or the ansa cervicalis nerve. The CPAP device 1908 is a separate device and is not an implanted device. The CPAP device 1908 comprises a pump and a mask worn over the mouth and nose of the patient and is operable to provide continuous positive air pressure to the patient to assist with breathing and to help regulate respiratory rate.

The neurostimulator implant system 1900 also includes a controller 1902. The controller 1902 may be provided in an external device, for example the external device 302, 510 shown in FIG. 3A to FIG. 6. Alternatively, the controller 1902 may be a part of at least one of the neurostimulator implants, or may be provided in a separate device, for example a computing device in communication with the neurostimulator implants 1904, 1906 and the CPAP device 1908.

The neurostimulator implant system 1900 also includes a sensor 1910. The sensor 1910 is arranged to detect at least one indicator of sleep apnea. The sensor 1910 may include one or more sensors. In examples, the sensor 1910 is arranged to detect at least one of: respiratory rate, heart rate, mandibular movement(s), thoracic movement(s), diaphragm movement(s), oxygen saturation, and/or EMG. The sensor 1910 may be provided in one or more of the neurostimulator implants 1904, 1906, and/or the sensor 1910 may be provided in the CPAP device 1908, and/or the sensor 1910 may be provided in the external device 302, 510, and/or the sensor 1910 may be a separate sensor that may be attachable to the patient at a different location.

The controller 1902 is configured to operate the hypoglossus neurostimulator implant 1904, and/or phrenic nerve neurostimulator implant 1906, and/or the CPAP device 1908 in response to an apneic event detected by the sensor 1910. The controller 1902 may be configured to determine, based on data provided by the sensor 1910, whether an apneic event can be classified as OSA, CSA, or a combination of OSA and CSA. The controller 1902 may be configured to operate one or more of the hypoglossus neurostimulator implant 1904, the phrenic nerve neurostimulator implant 1906, and/or the CPAP device 1908 based on the classification of the apneic event.

For example, in the event that the controller 1902 detects an OSA event, the controller 1902 may be configured to operate the hypoglossus neurostimulator implant 1904 to stimulate the left and/or right genioglossus nerve branch 106. Such simulation can cause protraction of the tongue and alleviate the OSA.

In a further example, in the event that the controller 1902 detects a CSA event, the controller 1902 may be configured to operate the phrenic nerve neurostimulator implant 1906 to stimulate the left phrenic nerve 602 and/or the right phrenic nerve 604 as shown in FIG. 6 (and/or the ansa cervicalis nerve). Such stimulation may alleviate the CSA.

Additionally or alternatively, in the event that the controller 1902 detects an OSA event, the controller 1902 may be configured to operate the CPAP device 1908 to provide positive air pressure and regulate the patient's respiratory rate. Such positive air pressure may alleviate the OSA.

In a further example, in the event that the controller 1902 detects a combined OSA and CSA event, the controller 1902 may be configured to operate the hypoglossus neurostimulator implant 1904 to stimulate at least one of the right hypoglossal nerve 402 and the left hypoglossal nerve 404, and also operate the phrenic nerve neurostimulator implant 1906 to stimulate at least one of the left phrenic nerve 602 and the right phrenic nerve 604 (and/or the ansa cervicalis nerve). Such combined stimulation may alleviate the apneic event. Additionally or alternatively, the controller 1902 may be configured to control the CPAP device 1908 to additionally provided continuous positive air pressure to the patient to further alleviate the apneic event.

Advantageously, the neurostimulator implant system 1900 can thereby used the information from the one or more sensors 1910 to determine the onset or presence of sleep apnea, and to determine a classification of the sleep apnea (i.e., OSA, CSA, or a combination of OSA and CSA). Different forms and combinations of neural stimulation and positive air pressure treatment can then be provided to alleviate the sleep apnea.

In examples, the treatment delivered by the neurostimulator implant system 1900 can be modulated with respect to amplitude and/or frequency. In particular, in some examples, the CPAP device 1908 and/or the hypoglossus neurostimulator implant 1904 and/or the phrenic nerve neurostimulator implant 1906 may be operated via sequenced pulse trains, whereby each sequence, pulse train and pulse (within each pulse train) can be individually modulated according to a determined treatment. In addition, the start time, stop time and any delay time can be adjusted as required based on the information provided by the sensor 1910. Adjustments to the treatment may be made manually and/or automatically in response to the information provided by the sensor 1910.

In some examples, the sensor 1910 detects mandibular movements (e.g., rotational movements of the mandible) and the neurostimulator implant system 1900 configures treatment provided the hypoglossus neurostimulator implant 1904, phrenic nerve neurostimulator implant 1906 and/or CPAP device 1908 based on the detected mandibular movements. In such examples, the sensor 1910 may comprise a gyroscope and/or accelerometer and/or magnetometer, which may be in one or more of the hypoglossus neurostimulator implant 1904 or phrenic nerve neurostimulator implant 1906, and/or in an external device, and/or in a separate sensor unit. The treatment may be configured with different period and/or frequency and/or amplitude, and/or duration, and/or a different combination of positive air pressure and neural stimulation, based on the detected mandibular movements.

In some examples, the sensor 1910 is arranged to detect other movements, for example thorax movements and/or head movements, in a similar manner as described above. The treatment can be configured, based on the detected thorax and/or head movements, in the same way as described above, specifically by adapting the period, frequency and/or amplitude, and/or duration, and/or a different combination of positive air pressure and neural stimulation, based on the detected movement.

In some examples, the sensor 1910 is arranged to detect oxygen saturation and/or EMG. In such examples, the treatment can be configured, based on the oxygen saturation and/or EMG, by adapting the period and/or frequency and/or amplitude, and/or duration, and/or a different combination of positive air pressure and neural stimulation, based on the detected oxygen saturation and/or EMG.

In some examples, the sensor 1910 is arranged to detect a respiratory rate of the patient, in particular lapses in the respiratory rate. In such examples, the treatment can be configured, based on the detected respiratory rate, in the same way as described above. In particular, the treatment may be configured with different period and/or frequency and/or amplitude, and/or duration, and/or a different combination of positive air pressure and neural stimulation, based on the detected respiratory rate. In some examples, the treatment (specifically the positive air pressure and/or neural stimulation) may be provided synchronously with the respiratory rate of the patient detected by the sensor 1910. In examples, the the treatment (specifically the positive air pressure and/or neural stimulation) may be provided at a predetermined respiratory rate in order to regulate the respiratory rate of the patient.

In some examples, the controller 1902 is configured to configure the treatment such that the CPAP device 1908 and/or the hypoglossus neurostimulator implant 1904 and/or the phrenic nerve neurostimulator implant 1906 are operated at specific phases of the respiration cycle. For example, one or more may be activated during an inhalation cycle and deactivated during exhalation, or vice versa. In some examples, the controller 1902 is configured to operate the hypoglossus neurostimulator implant 1904 and/or phrenic nerve neurostimulator implant 1906 and/or CPAP device 1908 synchronously, either in phase with each other or out of phase with each other. For example, neural stimulation may be provided at the start of an inhalation cycle, and positive air pressure may be provided during the inhalation cycle.

In examples, the sensor 1910 is configured to continuously or intermittently detect one or more parameters of the patient, and the controller 1902 is configured to continuously or intermittently (re)configure the treatment being provided to the patient. This provides a closed-loop system for treating sleep apnea.

In examples, the controller 1902 may be configured to operate the hypoglossus neurostimulator implant 1904, the phrenic nerve neurostimulator implant 1906, and/or the CPAP device 1908 in response to a detected respiratory lapse, a fall of the detected respiratory rate below a threshold value, the detection of one or more indicators of sleep apnea (e.g., based on detected respiratory lapse, and/or mandibular, thorax and/or head movement, and/or oxygen saturation, and/or EMG).

In various examples described above a neurostimulator implant 200 provides neural stimulation to a nerve, specifically the genioglossus nerve branch 106 and/or phrenic nerve 502 and/or ansa cervicalis nerve. The stimulation provided by the electrodes 206a, 206b, 206c, 206d of the neurostimulator implant 200 is based on a stimulation signal generated by a signal generator within the housing portion 202. The stimulation signal characteristics may be determined by the signal generator in the neurostimulator implant 200, or they may be determined by an external controller and communicated to the neurostimulator implant 200.

The stimulation signal may include random signal generation, combining high and low frequencies. High and low frequency stimulation signals may be generated sequentially or simultaneously. The stimulation signal may have a frequency in a range of between 2 Hz and 1500 Hz, depending on whether the stimulation is directed at a muscle or a nerve. In one example, the stimulation signal may comprise a low frequency component for stimulating a muscle (e.g., the genioglossus muscle). In such an example, the low frequency component may have a frequency of between 2 Hz and 150 Hz. In examples, the stimulation signal may have a high frequency component for stimulating a nerve (e.g., the genioglossus nerve branch). In such an example, the high frequency component may have a frequency of between 20 Hz and 1500 Hz.

In some examples, the stimulation signal has a variable pulse width. The pulse width may be between 20 microseconds and 10,000 microseconds. The pulses may be charge balanced. The stimulation signal may be monopolar, bipolar, or a combination of monopolar and bipolar. An amplitude of the stimulation signal may be up to 10.5V amplitude, and up to 25 mA amplitude.

In examples, the electrode providing a stimulating signal to a nerve or muscle may be operated as an anode or as a cathode. The neurostimulator implant may be controllable to change between anodic stimulation and cathodic stimulation.

Throughout the description and claims of this specification, the words “comprise” and “contain” and variations of them mean “including but not limited to”, and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and, accordingly, all suitable modifications and equivalents falling within the scope of the disclosure may be resorted to.

At various places in the present specification, values may be disclosed in groups or in ranges. It is specifically intended that the description include each and every individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 16, 17, 18, 19, and 20. Real numbers are intended to be similarly inclusive, including values up to at least three decimal places.

Features, integers, characteristics or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Moreover, while illustrative embodiments have been described herein, the scope of any and all embodiments include equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A method of treating sleep apnea in a patient, the method comprising operating an implanted neurostimulator implant to stimulate a branch of the hypoglossal nerve.

2. The method of claim 1, wherein the method comprises operating the implanted neurostimulator implant to stimulate the genioglossus nerve branch and/or the genioglossus muscle.

3. The method of claim 2, wherein the implanted neurostimulator implant is operated to stimulate the genioglossus nerve branch with a first stimulation signal and to stimulate the genioglossus muscle with a second stimulation signal.

4. The method of claim 2 wherein the implanted neurostimulator implant is operated to stimulate the genioglossus muscle with a stimulation signal having a frequency of between about 2 Hz and about 150 Hz, for example between about 2 Hz and about 100 Hz, for example between about 2 Hz and about 50 Hz, for example between about 2 Hz and about 40 Hz, for example between about 2 Hz and about 30 Hz, for example between about 2 Hz and about 20 Hz, for example between about 2 Hz and about 15 Hz, for example between about 2 Hz and about 10 Hz.

5. The method of claim 4, wherein the stimulation signal stimulating the genioglossus muscle has a peak amplitude of between 0.1 mA and 10 mA, for example between 0.1 mA and 5 mA.

6. The method of claim 2 wherein the implanted neurostimulator implant is operated to stimulate the genioglossus nerve branch with a stimulation signal having a frequency of between about 20 Hz and about 1500 Hz, for example between about 20 Hz and about 1000 Hz, for example between about 20 Hz and about 500 Hz, for example between about 20 Hz and about 400 Hz, for example between about 20 Hz and about 300 Hz, for example between about 20 Hz and about 200 Hz, for example between about 20 Hz and about 150 Hz, for example between about and about 100 Hz.

7. The method of claim 2 wherein the stimulation of the genioglossus nerve branch and/or the genioglossus muscle comprises a stimulation signal having a plurality of pulses, each pulse having a pulse width of between 30 microseconds and 2,000 microseconds.

8. The method of claim 2 wherein the neurostimulator implant comprises an electrode lead having an electrode, and wherein the method comprises implanting the electrode lead in the genioglossus muscle proximal to the genioglossus nerve branch of the patient.

9. The method of claim 8, wherein the neurostimulator implant further comprises a housing portion, and wherein the electrode lead extends from the housing portion, and wherein the method comprises implanting the housing portion in a subcutaneous tissue of the patient.

10. The method of claim 1 further comprising transferring wireless power from an external device to the implanted neurostimulator implant.

11. The method of claim 10, wherein the wireless power transferred to the implanted neurostimulator implant comprises a frequency of between 300 MHz and 3 GHz, for example between 400 MHz and 2.5 GHz, for example between 433 MHz and 2.4 GHz.

12. The method of claim 1 further comprising detecting, by a sensor, at least one physiological parameter, for example at least one of:
a respiration rate of the patient,
an electromyograph, EMG, of the patient,
an electrocardiogram, ECG, of the patient,
an oxygen saturation of the patient,
a body temperature of the patient,
a heart rate of the patient,
a blood pressure of the of the patient.

13. The method of claim 1 further comprising, detecting, by a sensor, at least one movement of the patient, for example at least one of:
a respiratory movement of the patient,
a mandibular movement of the patient,
a thoracic movement of the patient,
a diaphragm movement of the patient.

14. The method of claim 1 further comprising detecting, by a sensor, a physiological parameter or movement of the patient indicative of sleep apnea, for example at least one of:
a lapse is respiratory rate,
an electromyograph, EMG, profile indicative of sleep apnea,
an electrocardiogram, ECG, profile indicative of sleep apnea,
a decrease in oxygen saturation,
an increase in body temperature,
an increase in heart rate,
an increase in blood pressure, or
a respiratory, mandibular, thoracic or diaphragm movement.

15. The method of claim 12 further comprising controlling the neurostimulator implant to stimulate the genioglossus nerve branch and/or the genioglossus muscle in response to the detected at least one physiological parameter of the patient and/or the detected at least one movement of the patient.

16. The method of claim 12 comprising detecting, by a sensor, a respiratory rate of the patient, and stimulating the genioglossus nerve branch and/or the genioglossus muscle synchronously with the detected respiratory rate.

17. The method of claim 16, comprising stimulating the genioglossus nerve branch and/or the genioglossus muscle at the start of an inhalation cycle of the patient.

18. The method of claim 12 further comprising determining, from the detected at least one physiological parameter and/or at least one movement of the patient, a classification of sleep apnea, in particular whether the detected sleep apnea comprises obstructive sleep apnea, OSA, central sleep apnea, CSA, or a combination of OSA and CSA.

19. The method of claim 18, further comprising operating a second implanted neurostimulator implant to stimulate a phrenic nerve and/or an ansa cervicalis nerve of the patient.

20. The method of claim 18 further comprising operating a continuous positive air pressure, CPAP, device to provide positive air pressure to the patient's airways.

21. The method of claim 19 further comprising controlling the or each neurostimulator implant, and optionally the CPAP device, based on the detected at least one physiological parameter of the patient and/or the detected at least one movement of the patient.

22. The method of claim 18 further comprising operating the neurostimulator implant and/or the second neurostimulator implant and/or the CPAP device based on the determined classification of sleep apnea.

23. The method of claim 22, comprising, when the classification of sleep apnea is determined to be obstructive sleep apnea, OSA, operating the neurostimulator device to stimulate the genioglossus nerve branch and/or the genioglossus muscle.

24. The method of claim 22, comprising, when the classification of sleep apnea is determined to be central sleep apnea, CSA, operating the second neurostimulator implant to stimulate the phrenic nerve and/or ansa cervicalis nerve.

25. The method of claim 22 comprising, when the classification of sleep apnea is determined to be obstructive sleep apnea, OSA, operating the CPAP device to provide positive air pressure to the patient.

26. The method of claim 22 comprising, if the classification of sleep apnea is determined to be a combination of obstructive sleep apnea and central sleep apnea, operating:
the neurostimulator device to stimulate the genioglossus nerve branch and genioglossus muscle, and
at least one of:
the second neurostimulator implant to stimulate the phrenic nerve and/or ansa cervicalis nerve,
and the CPAP device to provide positive air pressure to the patient.

27. The method of claim 12 wherein the sensor is integrated with the neurostimulator implant.

28. A system for treating sleep apnea of a patient, the system comprising a neurostimulator implant implantable proximal to a branch of the hypoglossal nerve of the patient and operable to stimulate the branch of the hypoglossal nerve.

29. The system of claim 28, wherein the implanted neurostimulator implant is operable to stimulate the genioglossus nerve branch and/or the genioglossus muscle of the patient.

30. The system of claim 29, wherein the neurostimulator implant comprises an electrode that is implantable within the genioglossus muscle proximal to a genioglossus nerve branch of the patient, and a signal generator for generating a stimulation signal to stimulate the genioglossus nerve branch and/or the genioglossus muscle of the patient.

31. The system of claim 29, wherein the neurostimulator implant is a battery-less implant and comprises a wireless power receiver.

32. The system of claim 31, further comprising an external device comprising a wireless power transmitter for transmitting wireless power to the neurostimulator implant when the neurostimulator implant is implanted in the genioglossus muscle.

33. The system of claim 28 further comprising a sensor arranged to detect at least one physiological parameter of the patient and/or at least one movement of the patient.

34. The system of claim 33, wherein the at least one physiological parameter comprises one or more of:
a respiration rate of the patient,
an electromyograph, EMG, of the patient,
an electrocardiogram, ECG, of the patient,
an oxygen saturation of the patient,
a body temperature of the patient,
a heart rate of the patient,
a blood pressure of the of the patient.

35. The system of claim 33 wherein the at least one movement of the patient comprises at least one of:
a respiratory movement of the patient,
a mandibular movement of the patient,
a thoracic movement of the patient,
a diaphragm movement of the patient.

36. The system of claim 33 further comprising a controller configured to determine the onset or occurrence of sleep apnea based on the detected physiological parameter and/or the detected movement of the patient, for example at least one of:
a lapse is respiratory rate,
an electromyograph, EMG, profile indicative of sleep apnea,
an electrocardiogram, ECG, profile indicative of sleep apnea,
a decrease in oxygen saturation,
an increase in body temperature,
an increase in heart rate,
an increase in blood pressure, or
a respiratory, mandibular, thoracic or diaphragm movement.

37. The system of claim 28 further comprising a second neurostimulator implant implantable to stimulate a phrenic nerve and/or an ansa cervicalis nerve of the patient.

38. The system of claim 28 further comprising a continuous positive air pressure, CPAP, device operable to provide positive air pressure to the patient.

39. The system of claim 28 further comprising a controller configured to control the or each neurostimulator implant, and optionally the CPAP device, based on the detected at least one physiological parameter of the patient and/or the detected at least one movement of the patient.

40. The system of claim 28 wherein the controller is configured to determine, based on the detected at least one physiological parameter and/or the at least one movement of the patient, a classification of sleep apnea, in particular whether the detected sleep apnea comprises obstructive sleep apnea, OSA, central sleep apnea, CSA, or a combination of OSA and CSA.

41. The system of claim 40, wherein the controller is configured to operate the neurostimulator implant and/or the second neurostimulator implant and/or the CPAP device based on the determined classification of sleep apnea.

42.-76. (canceled)
